(19) United States
(12) Patent Application Publication
(10) Pub. No.: US 2011/0077270 A1
(43) Pub. Date: Mar. 31, 2011

(54) Compositions and Methods for Treating Ocular Inflammation with Lower Risk of Increased Intraocular Pressure

(76) Inventors: Bruce A. Pfeffer, Fairport, NY (US); Mercedes Salvador-Silva, Rochester, NY (US); Charu A. DeWitt, Pittsford, NY (US); Keith W. Ward, Ontario, NY (US); Francisco J. Lopez, Victor, NY (US)

(21) Appl. No.: 12/732,460

(22) Filed: Mar. 26, 2010

Related U.S. Application Data
(60) Provisional application No. 61/171,181, filed on Apr. 21, 2009.

(51) Int. Cl. A61K 31/4709; A61P 29/00; A61P 27/02; A61P 3/10

(52) U.S. Cl. 514/313

(57) ABSTRACT

A composition for treating or controlling an ocular disease or condition comprises a dissociated glucocorticoid receptor agonist (“DIGRA”), which disease or condition has an etiology, or results, in inflammation. The composition can optionally include an anti-inflammatory agent, an anti-infective agent, or both. The composition can be formulated for topical application, injection, or implantation in an affected eye to treat or control the ocular inflammatory disease or condition.

### CROSS REFERENCE

This application claims the benefit of Provisional Patent Application No. 61/171,181 filed Apr. 21, 2009 which is incorporated by reference herein.

### BACKGROUND OF THE INVENTION

The present invention relates to compositions and methods for treating or controlling ocular inflammation. In particular, the present invention relates to compositions that comprise dissociated glucocorticoid receptor agonists (“DIGRAs”) and methods for the treatment or control of ocular inflammation using such compositions, which compositions and methods provide lower risk of increased intraocular pressure.

Many anterior- and posterior-segment ocular disorders have etiology in inflammation. For example, various studies have established or strongly suggested that diseases such as corneal edema, anterior and posterior uveitis, pterygium, corneal diseases, dry eye, conjunctivitis, allergy- and laser-induced exudation, macular degeneration, macular edema, diabetic retinopathy, and age-related macular degeneration have a root cause in inflammation. See; e.g., I. Kim et al., Biol. Chem., Vol. 276, No. 10, 7614 (2001); A. M. Joussen et al., FASEB J., Vol. 18, 1450 (2004); S. C. Pflugfelder, Am. J. Ophthalmol., Vol. 137, 337 (2004). In addition, tumor necrosis factor-α (“TNF-α”), a proinflammatory cytokine, has recently been identified to be a mediator of retinal ganglion cell (“RGC”) death. TNF-α and TNF-α receptor-1 are up-regulated in experimental rat models of glaucoma. In vitro studies have further identified that TNF-α-mediated RGC death involves the activation of both receptor-mediated caspase cascade and mitochondria-mediated caspase-dependent and caspase-independent components of cell death cascade. G. Tezel and X. Yang, Expt'l Eye Res., Vol. 81, 207 (2005). Moreover, TNF-α and its receptor were found in greater amounts in retina sections of glaucomatous eyes than in control eyes of age-matched normal donors. G. Tezel et al., Invest. Ophthalmol. & Vis. Sci., Vol. 42, No. 8, 1787 (2001). Therefore, there has been growing evidence that glaucoma may have a root cause in chronic inflammation. Failure to control the insult-induced immune response can result in autoimmune pathogenesis and likely initiates or sustains glaucomatous neurodegeneration in many patients.

Glucocorticoids (“GC,” also herein referred to as corticosteroids) are often prescribed to treat a variety of ocular conditions having an inflammatory or neovascular component, such as macular edema, “wet” age-related macular degeneration, uveitis, and complications of surgery. The therapeutic benefit of GC is due to pleiotropic modulation and mobilization of multiple intracellular signaling pathways, encompassing predominantly transrepressive effects of the steroid-nuclear receptor complex that interfere with elements governing transcription of selected genes. One of the adverse events commonly associated with glucocorticoid therapy, regardless of route of administration, is an elevation of intraocular pressure (“IOP”) that may lead to glaucoma, a side-effect assumed to result from transactivation of a gene or genes unrelated to the indication being treated. Some patients receiving ocular GC may exhibit no effect, while others, classified as responders, demonstrate a range of documented increases in IOP, attributed to several risk factors, including age, history of primary open-angle glaucoma (“POAG”), and genetic predisposition.

POAG is characterized by high TOP, resulting from impaired efflux of aqueous fluid through the trabecular meshwork (“TM”). Since the juxtacanalicular region (“JCT”) of the TM abutting the inner wall endothelium of Schlemm's canal is the likely site of resistance to outflow under normal physiological conditions, structural and biochemical changes in the JCT would be expected to affect IOP. A feature shared by both POAG and steroid-induced glaucoma is the accumulation of extracellular matrix (“ECM”) and other material (“plaque”) in the JCT, consisting of abnormal aggregates of macromolecules obstructing the outflow pathway and raising IOP. As with many other non-ocular cells and tissues that have been examined, the TM is susceptible to GC-induced ECM changes, demonstrated experimentally and in clinical samples.

Myocilin is a protein normally detected in eye tissues, and whose constitutive expression is most pronounced in the TM, both intra- and extracellularly. The discovery that mutations in the myocilin gene (MYOC) give rise to selected forms of POAG and juvenile open-angle glaucoma eventually directed attention to the roles of wild-type myocilin in eye health and disease. An apparently unique—and diagnostic—property of TM cells in vitro and in situ is the overexpression of myocilin in response to GC. The precise functional role of myocilin is not understood, but GC-enhanced TM expression of myocilin has raised the possibility that this protein has an etiological role in steroid-induced glaucoma. Besides effects on TM cell internal structure and function, as assessed in organ cultured material, GC treatment may induce excessive myocilin synthesis and secretion by these cells, culminating with deposition of the protein in the ECM of the outflow pathway, and hence, elevating IOP. Pharmacologic doses of dexamethasone (“DEX”) elicit elevated expression of myocilin in cultured TM cells from normal human donors, shown by analysis of myocilin mRNA or through immunochemical detection of soluble myocilin released into culture media. Irrespective of whether or not these changes underlie a direct role for myocilin in the pathophysiology of any form of glaucoma, drug-induced elevations of this protein could be considered a surrogate indicator of risk for secondary glaucoma.

There is considerable interest in non-steroidal GC receptor (GR) agonists that, by virtue of their structures and of the specific conformational changes they generate upon binding to the GR, may exhibit partial dissociation with respect to transactivation and transrepression of selected genes normally affected by GCs. Molecules with these distinct biochemical profiles may offer an improved clinical safety profile compared to steroidal GR agonists routinely used in the clinic. Human TM cells have been widely employed as an in vitro model to study responses to GCs.

Therefore, there is a continued need to provide improved pharmaceutical compounds, compositions, and methods to treat or control ocular inflammatory diseases, conditions, or disorders that provide lower risk of increased IOP than a composition and a method using a prior-art glucocorticoid used to treat or control the same diseases, conditions, or disorders.

### SUMMARY OF THE INVENTION

As used herein, the term “control” also includes reduction, alleviation, amelioration, and prevention.

In general, the present invention provides compounds, compositions, or methods for treating or controlling an ocular inflammatory disease, disease, condition, or disorder. Such an inflammatory disease, condition, or disorder has etiology in, or produce, inflammation.

In one aspect, the compounds, compositions, methods of the present invention provides a lower risk of increased IOP than a composition and method using a prior-art GC to treat or control the same diseases, conditions, or disorders.

In another aspect, said ocular inflammatory diseases, conditions, or disorders of the anterior segment include uveitis (including anterior uveitis, posterior uveitis, and panuveitis), keratitis, conjunctivitis, keratoconjunctivitis (including vernal keratoconjunctivitis (or “VKC”) and atopic keratoconjunctivitis), corneal ulcer, corneal edema, sterile corneal infiltrates, anterior scleritis, episcleritis, blepharitis, and post-operative (or post-surgical) ocular inflammation resulting from procedures such as photorefractive keratectomy, cataract removal surgery, intraocular lens (“IOL”) implantation, laser-assisted in situ keratomileusis (“LASIK”), conductive keratoplasty, radial keratotomy, dry eye, macular degeneration, macular edema, diabetic retinopathy, age-related macular degeneration (including the wet and dry forms), and glaucoma (including all types of glaucoma).

In still another aspect, such inflammatory diseases, conditions, or disorders result from an infection caused by bacteria, viruses, fungi, or protozoans.

In yet another aspect, the compositions comprise at least a mimetic of a glucocorticoid for treating or controlling such diseases, conditions, or disorders.

In yet another aspect, a pharmaceutical composition for treating or controlling an inflammatory disease, condition, or disorder comprises at least a dissociated glucocorticoid receptor agonist (“DIGRA”), a prodrug, or a pharmaceutically acceptable salt or ester thereof.

In yet another aspect, a pharmaceutical composition of the present invention comprises an ophthalmic topical formulation; injectable formulation; or implantable formulation, system, or device.

In still another aspect, such a formulation, system, or device is applied or provided to the anterior segment of the eye.

In still another aspect, such a formulation, system, or device is applied or provided to the posterior segment of the eye.

In a further aspect, said increased IOP is demonstrated in vitro or in vivo.

In yet another aspect, said increased IOP results from an increased resistance of fluid outflow through the trabecular meshwork.

In still another aspect, such increased resistance results from increased expression and accumulation of myocilin in the trabecular meshwork.

In another aspect, the present invention provides a method for treating or controlling an inflammatory disease, condition, or disorder of the anterior segment. The method comprises administering a composition comprising a DIGRA, a prodrug thereof, or a pharmaceutically acceptable salt or ester thereof to an affected eye of a subject in need of such treatment or control.

Other features and advantages of the present invention will become apparent from the following detailed description and claims.

### BRIEF DESCRIPTION OF THE DRAWINGS

FIGS. 1A-1F show the effects of BOL-303242-X and dexamethasone on the IL-1β-stimulated production of Il-6, IL-7, TGF-α, TNF-α, VGEF, and MCP-1 in human corneal epithelium cells (“HCECs”) at p<0.05.

FIG. 2 shows the effects of BOL-303242-X and dexamethasone on the IL-1β-stimulated production of G-CSF in HCECs at p<0.05.

FIGS. 3A-1C show the effects of BOL-303242-X and dexamethasone on the IL-1β-stimulated production of GM-CSF, IL-8, and RANTES in HCECs at p<0.05.

In the foregoing figures, “*” denotes comparison to control, and “**” to IL-1β.

FIG. 4 shows a comparison of effects of BOL-303242-X (SEGRA) vs. DEX on myocilin protein in CM of monkey TM cells. Myocilin protein band densities are represented for a single TM strain in one study. *P<0.05 vs. the vehicle control. † P<0.05 vs. same dose of DEX. Open bar represents vehicle-treated cells. Two-way ANOVA followed by the contrast procedure on logarithmically transformed data. Data are presented as geometric means±SE estimated using the Taylor series expansion.

FIG. 5 shows representative quantitative real-time RT-PCR results for a single strain of monkey TM cells, from a dose-response study comparing the effects either of BOL-303242-X with DEX, on myocilin mRNA expression. *P<0.05 vs. vehicle control (open columns). † P<0.05 for either BOL-303242-X or PA, vs. DEX at the same concentration tested. Two-way ANOVA followed by the contrast procedure on logarithmically transformed data (SEGRA vs. DEX) or transformed data elevated to the power 0.2 (PA vs. DEX). Data are presented as geometric means±SE estimated using the Taylor series expansion.

### DETAILED DESCRIPTION OF THE INVENTION

As used herein, a dissociated glucocorticoid receptor agonist (“DIGRA”) is a compound that is capable of binding to the glucocorticoid receptor (which is a polypeptide) and, upon binding, is capable of producing differentiated levels of transrepression and transactivation of gene expression. A compound that binds to a polypeptide is sometimes herein referred to as a ligand.

As used herein, the term “alkyl” or “alkyl group” means a linear- or branched-chain saturated aliphatic hydrocarbon monovalent group, which may be unsubstituted or substituted. The group may be partially or completely substituted with halogen atoms (F, Cl, Br, or I). Non-limiting examples of alkyl groups include methyl, ethyl, n-propyl, 1-methylethyl(isopropyl), n-butyl, n-pentyl, 1,1-dimethylethyl (t-butyl), and the like. It may be abbreviated as “Alk”.

As used herein, the term “alkenyl” or “alkenyl group” means a linear- or branched-chain aliphatic hydrocarbon monovalent radical containing at least one carbon-carbon double bond. This term is exemplified by groups such as ethenyl, propenyl, n-butenyl, isobutenyl, 3-methylbut-2-enyl, n-pentenyl, heptenyl, octenyl, decenyl, and the like.

As used herein, the term “alkynyl” or “alkynyl group” means a linear- or branched-chain aliphatic hydrocarbon monovalent radical containing at least one carbon-carbon triple bond. This term is exemplified by groups such as ethynyl, propynyl, n-butynyl, 2-butynyl, 3-methylbutynyl, n-pentynyl, heptynyl, octynyl, decynyl, and the like.

As used herein, the term “alkylene” or “alkylene group” means a linear- or branched-chain saturated aliphatic hydrocarbon divalent radical having the specified number of carbon atoms. This term is exemplified by groups such as methylene, ethylene, propylene, n-butylene, and the like, and may alternatively and equivalently be denoted herein as “-(alkyl)-”.

The term “alkenylene” or “alkenylene group” means a linear- or branched-chain aliphatic hydrocarbon divalent radical having the specified number of carbon atoms and at least one carbon-carbon double bond. This term is exemplified by groups such as ethenylene, propenylene, n-butenylene, and the like, and may alternatively and equivalently be denoted herein as “-(alkylenyl)-”.

The term “alkynylene” or “alkynylene group” means a linear- or branched-chain aliphatic hydrocarbon divalent radical containing at least one carbon-carbon triple bond. This term is exemplified by groups such as ethynylene, propynylene, n-butynylene, 2-butynylene, 3-methylbutynylene, n-pentynylene, heptynylene, octynylene, decynylene, and the like, and may alternatively and equivalently be denoted herein as “-(alkynyl)-”.

As used herein, the term “aryl” or “aryl group” means an aromatic carbocyclic monovalent or divalent radical of from 5 to 14 carbon atoms having a single ring (e.g., phenyl or phenylene), multiple condensed rings (e.g., naphthyl or anthranyl), or multiple bridged rings (e.g., biphenyl). Unless otherwise specified, the aryl ring may be attached at any suitable carbon atom which results in a stable structure and, if substituted, may be substituted at any suitable carbon atom which results in a stable structure. Non-limiting examples of aryl groups include phenyl, naphthyl, anthryl, phenanthryl, indanyl, indenyl, biphenyl, and the like. It may be abbreviated as “Ar”.

The term “heteroaryl” or “heteroaryl group” means a stable aromatic 5- to 14-membered, monocyclic or polycyclic monovalent or divalent radical, which may comprise one or more fused or bridged ring(s), preferably a 5- to 7-membered monocyclic or 7- to 10-membered bicyclic radical, having from one to four heteroatoms in the ring(s) independently selected from nitrogen, oxygen, and sulfur, wherein any sulfur heteroatoms may optionally be oxidized and any nitrogen heteroatom may optionally be oxidized or be quaternized. Unless otherwise specified, the heteroaryl ring may be attached at any suitable heteroatom or carbon atom which results in a stable structure and, if substituted, may be substituted at any suitable heteroatom or carbon atom which results in a stable structure. Non-limiting examples of heteroaryls include furanyl, thienyl, pyrrolyl, oxazolyl, thiazolyl, imidazolyl, pyrazolyl, isoxazolyl, isothiazolyl, oxadiazolyl, triazolyl, tetrazolyl, thiadiazolyl, pyridinyl, pyridazinyl, pyrimidinyl, pyrazinyl, triazinyl, indolizinyl, azaindolizinyl, indolyl, azaindolyl, diazaindolyl, dihydroindolyl, dihydroazaindoyl, isoindolyl, azaisoindolyl, benzofuranyl, furanopyridinyl, furanopyrimidinyl, furanopyrazinyl, furanopyridazinyl, dihydrobenzofuranyl, dihydrofuranopyridinyl, dihydrofuranopyrimidinyl, benzothienyl, thienopyridinyl, thienopyrimidinyl, thienopyrazinyl, thienopyridazinyl, dihydrobenzothienyl, dihydrothienopyridinyl, dihydrothienopyrimidinyl, indazolyl, azaindazolyl, diazaindazolyl, benzimidazolyl, imidazopyridinyl, benzthiazolyl, thiazolopyridinyl, thiazolopyrimidinyl, benzoxazolyl, benzoxazinyl, benzoxazinonyl, oxazolopyridinyl, oxazolopyrimidinyl, benzisoxazolyl, purinyl, chromanyl, azachromanyl, quinolizinyl, quinolinyl, dihydroquinolinyl, tetrahydroquinolinyl, isoquinolinyl, dihydroisoquinolinyl, tetrahydroisoquinolinyl, cinnolinyl, azacinnolinyl, phthalazinyl, azaphthalazinyl, quinazolinyl, azaquinazolinyl, quinoxalinyl, azaquinoxalinyl, naphthyridinyl, dihydronaphthyridinyl, tetrahydronaphthyridinyl, pteridinyl, carbazolyl, acridinyl, phenazinyl, phenothiazinyl, and phenoxazinyl, and the like.

The term “heterocycle”, “heterocycle group”, “heterocyclyl”, “heterocyclyl group”, “heterocyclic”, or “heterocyclic group” means a stable non-aromatic 5- to 14-membered monocyclic or polycyclic, monovalent or divalent, ring which may comprise one or more fused or bridged ring(s), preferably a 5- to 7-membered monocyclic or 7- to 10-membered bicyclic ring, having from one to three heteroatoms in at least one ring independently selected from nitrogen, oxygen, and sulfur, wherein any sulfur heteroatoms may optionally be oxidized and any nitrogen heteroatom may optionally be oxidized or be quaternized. As used herein, a heterocyclyl group excludes heterocycloalkyl, heterocycloalkenyl, and heterocycloalkynyl groups. Unless otherwise specified, the heterocyclyl ring may be attached at any suitable heteroatom or carbon atom which results in a stable structure and, if substituted, may be substituted at any suitable heteroatom or carbon atom which results in a stable structure. Non-limiting examples of heterocycles include pyrrolinyl, pyrrolidinyl, pyrazolinyl, pyrazolidinyl, piperidinyl, morpholinyl, thiomorpholinyl, piperazinyl, tetrahydropyranyl, tetrahydrothiopyranyl, tetrahydrofuranyl, hexahydropyrimidinyl, hexahydropyridazinyl, and the like.

The term “cycloalkyl” or “cycloalkyl group” means a stable aliphatic saturated 3- to 15-membered monocyclic or polycyclic monovalent radical consisting solely of carbon and hydrogen atoms which may comprise one or more fused or bridged ring(s), preferably a 5- to 7-membered monocyclic or 7- to 10-membered bicyclic ring. Unless otherwise specified, the cycloalkyl ring may be attached at any carbon atom which results in a stable structure and, if substituted, may be substituted at any suitable carbon atom which results in a stable structure. Exemplary cycloalkyl groups include cyclopropyl, cyclobutyl, cyclopentyl, cyclohexyl, cycloheptyl, cyclooctyl, cyclononyl, cyclodecyl, norbornyl, adamantyl, tetrahydronaphthyl (tetralin), 1-decalinyl, bicyclo[2.2.2]octanyl, 1-methylcyclopropyl, 2-methylcyclopentyl, 2-methylcyclooctyl, and the like.

The term “cycloalkenyl” or “cycloalkenyl group” means a stable aliphatic 5- to 15-membered monocyclic or polycyclic monovalent radical having at least one carbon-carbon double bond and consisting solely of carbon and hydrogen atoms which may comprise one or more fused or bridged ring(s), preferably a 5- to 7-membered monocyclic or 7- to 10-membered bicyclic ring. Unless otherwise specified, the cycloalkenyl ring may be attached at any carbon atom which results in a stable structure and, if substituted, may be substituted at any suitable carbon atom which results in a stable structure. Exemplary cycloalkenyl groups include cyclopentenyl, cyclohexenyl, cycloheptenyl, cyclooctenyl, cyclononenyl, cyclodecenyl, norbornenyl, 2-methylcyclopentenyl, 2-methylcyclooctenyl, and the like.

The term “cycloalkynyl” or “cycloalkynyl group” means a stable aliphatic 8- to 15-membered monocyclic or polycyclic monovalent radical having at least one carbon-carbon triple bond and consisting solely of carbon and hydrogen atoms which may comprise one or more fused or bridged ring(s), preferably a 8- to 10-membered monocyclic or 12- to 15-membered bicyclic ring. Unless otherwise specified, the cycloalkynyl ring may be attached at any carbon atom which results in a stable structure and, if substituted, may be substituted at any suitable carbon atom which results in a stable structure. Exemplary cycloalkynyl groups include cyclooctynyl, cyclononynyl, cyclodecynyl, 2-methylcyclooctynyl, and the like.

The term “carbocycle” or “carbocyclic group” means a stable aliphatic 3- to 15-membered monocyclic or polycyclic monovalent or divalent radical consisting solely of carbon and hydrogen atoms which may comprise one or more fused or bridged rings, preferably a 5- to 7-membered monocyclic or 7- to 10-membered bicyclic ring. Unless otherwise specified, the carbocycle may be attached at any carbon atom which results in a stable structure and, if substituted, may be substituted at any suitable carbon atom which results in a stable structure. The term comprises cycloalkyl (including spiro cycloalkyl), cycloalkylene, cycloalkenyl, cycloalkenylene, cycloalkynyl, and cycloalkynylene, and the like.

The terms “heterocycloalkyl”, “heterocycloalkenyl”, and “heterocycloalkynyl” mean cycloalkyl, cycloalkenyl, and cycloalkynyl group, respectively, having at least a heteroatom in at least one ring, respectively.

Glucocorticoids (“GCs”) are among the most potent drugs used for the treatment of allergic and chronic inflammatory diseases or of inflammation resulting from infections. However, as mentioned above, long-term treatment with GCs is often associated with numerous adverse side effects, such as diabetes, osteoporosis, hypertension, glaucoma, or cataract. These side effects, like other physiological manifestations, are results of aberrant expression of genes responsible for such diseases. Research in the last decade has provided important insights into the molecular basis of GC-mediated actions on the expression of GC-responsive genes. GCs exert most of their genomic effects by binding to the cytoplasmic GC receptor (“GR”). The binding of GC to GR induces the translocation of the GC-GR complex to the cell nucleus where it modulates gene transcription either by a positive (transactivation) or negative (transrepression) mode of regulation. There has been growing evidence that both beneficial and undesirable effects of GC treatment are the results of undifferentiated levels of expression of these two mechanisms; in other words, they proceed at similar levels of effectiveness. Although it has not yet been possible to ascertain the most critical aspects of action of GCs in chronic inflammatory diseases, there has been evidence that it is likely that the inhibitory effects of GCs on cytokine synthesis are of particular importance. GCs inhibit the transcription, through the transrepression mechanism, of several cytokines that are relevant in inflammatory diseases, including IL-1β (interleukin-1β, IL-2, IL-3, IL-6, IL-11, TNF-α (tumor necrosis factor-α), GM-CSF (granulocyte-macrophage colony-stimulating factor), and chemokines that attract inflammatory cells to the site of inflammation, including IL-8, RANTES, MCP-1 (monocyte chemotactic protein-1), MCP-3, MCP-4, MIP-1α (macrophage-inflammatory protein-1α), and eotaxin. P. J. Barnes, Clin. Sci., Vol. 94, 557-572 (1998). On the other hand, there is persuasive evidence that the synthesis of lκB kinases, which are proteins having inhibitory effects on the NF-κB proinflammatory transcription factors, is increased by GCs. These proinflammatory transcription factors regulate the expression of genes that code for many inflammatory proteins, such as cytokines, inflammatory enzymes, adhesion molecules, and inflammatory receptors. S. Wissink et al., Mol. Endocrinol., Vol. 12, No. 3, 354-363 (1998); P. J. Barnes and M. Karin, New Engl. J. Med., Vol. 336, 1066-1077 (1997). Thus, both the transrepression and transactivation functions of GCs directed to different genes produce the beneficial effect of inflammatory inhibition. On the other hand, steroid-induced diabetes and glaucoma appear to be produced by the transactivation action of GCs on genes responsible for these diseases. H. Schäcke et al., Pharmacol. Ther., Vol. 96, 23-43 (2002). Thus, while the transactivation of certain genes by GCs produces beneficial effects, the transactivation of other genes by the same GCs can produce undesired side effects. Therefore, it is very desirable to provide pharmaceutical compounds and compositions that produce differentiated levels of transactivation and transrepression activity on GC-responsive genes to treat or control inflammatory diseases, conditions, or disorders, especially chronic inflammation.

In general, the present invention provides compounds, compositions, or methods for treating or controlling ophthalmic inflammatory diseases, conditions, or disorders (of the anterior segment and/or posterior segment) in a subject. Such inflammatory diseases, conditions, or disorders have etiology in, or produce, inflammation.

In one aspect, the compounds and compositions of the present invention cause a lower level of at least an adverse side effect than a composition comprising at least a prior-art glucocorticoid used to treat or control the same diseases, conditions, or disorders.

Ocular inflammatory pathways commence with the triggering of the arachidonic acid cascade. This cascade is triggered either by mechanical stimuli (such as the case of unavoidable surgically-inflicted trauma) or by chemical stimuli (such as foreign substances (e.g., components of disintegrated pathogenic microorganisms) or allergens). Prostaglandins are generated in most tissues by activation of the arachidonic acid pathway. Phospholipids in the damaged cell membrane are the substrate for phospholipase A to generate arachidonic acid and, in turn, the cyclooxygenase (“COX”) and lipoxygenase enzymes act on arachidonic acid to produce a family of pro-inflammatory prostaglandins, thromboxanes, and leukotrienes. These pro-inflammatory compounds recruit more immune cells (such as macrophages and neutrophils) to the site of injury, which then produce a greater amount of other pro-inflammatory cytokines and can further amplify the inflammation.

Cataract surgery with intraocular lens (“IOL”) implantation and glaucoma filtering microsurgery (trabeculectomy) are among the common ophthalmic surgical operations. These procedures are usually associated with some post-operative inflammation. The use of anti-inflammatory agents post-operatively can rapidly resolve this event to relieve the patient from pain, discomfort, visual impairment, and to reduce the risk of further complications (such as the onset of cystoid macular edema).

Thus, in one aspect, the present invention provides compounds or compositions for treating or controlling inflammatory diseases, conditions, or disorders of the anterior segment in a subject, wherein such inflammatory diseases, conditions, or disorders result from an infection caused by bacteria, viruses, fungi, protozoans, or combinations thereof.

In another aspect, such infection comprises an ocular infection.

In another aspect, such inflammatory diseases, conditions, or disorders of the anterior segment result from the physical trauma of ocular surgery.

In still another aspect, said inflammatory diseases, conditions, or disorders of the anterior segment include anterior uveitis (including; e.g., iritis and iridocyclitis), keratitis, conjunctivitis, keratoconjunctivitis (including vernal keratoconjunctivitis (or “VKC”) and atopic keratoconjunctivitis), corneal ulcer, corneal edema, sterile corneal infiltrates, anterior scleritis, episcleritis, blepharitis, and post-operative (or post-surgical) ocular inflammation resulting from procedures such as photorefractive keratectomy, cataract removal surgery, intraocular lens (“IOL”) implantation, laser-assisted in situ keratomileusis (“LASIK”), conductive keratoplasty, and radial keratotomy.

In yet another aspect, the compositions comprise at least a mimetic of a glucocorticoid for treating or controlling such diseases, conditions, or disorders.

In still another aspect, a level of said at least an adverse side effect is determined in vivo or in vitro. For example, a level of said at least an adverse side effect is determined in vitro by performing a cell culture and determining the level of a biomarker associated with said side effect. Such biomarkers can include proteins (e.g., enzymes), lipids, sugars, and derivatives thereof that participate in, or are the products of, the biochemical cascade resulting in the adverse side effect. Representative in vitro testing methods are further disclosed hereinbelow.

In still another aspect, said at least an adverse side effect is selected from the group consisting of glaucoma, cataract, hypertension, hyperglycemia, hyperlipidemia (increased levels of triglycerides), and hypercholesterolemia (increased levels of cholesterol).

In yet another embodiment, a level of said at least an adverse side effect is determined at about one day after said composition is first administered to, and are present in, said subject. In another embodiment, a level of said at least an adverse side effect is determined about 14 days after said composition is first administered to, and are present in, said subject. In still another embodiment, a level of said at least an adverse side effect is determined about 30 days after said composition is first administered to, and are present in, said subject. Alternatively, a level of said at least an adverse side effect is determined about 2, 3, 4, 5, or 6 months after said compounds or compositions are first administered to, and are present in, said subject.

In another aspect, said at least a prior-art glucocorticoid used to treat or control the same diseases, conditions, or disorders is administered to said subject at a dose and a frequency sufficient to produce an equivalent beneficial effect on said condition to a composition of the present invention after about the same elapsed time.

In still another aspect, said at least a prior-art glucocorticoid is selected from the group consisting of 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortarnate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, their physiologically acceptable salts, combinations thereof, and mixtures thereof. In one embodiment, said at least a prior-art glucocorticoid is selected from the group consisting of dexamethasone, prednisone, prednisolone, methylprednisolone, medrysone, triamcinolone, loteprednol etabonate, physiologically acceptable salts thereof, combinations thereof, and mixtures thereof. In another embodiment, said at least a prior-art glucocorticoid is acceptable for ophthalmic uses.

In one aspect, the compounds, compositions, methods of the present invention provides a lower risk of increased IOP than a composition and method using a prior-art GC to treat or control the same diseases, conditions, or disorders.

In another aspect, said ocular inflammatory diseases, conditions, or disorders of the anterior segment include uveitis (including anterior uveitis, posterior uveitis, and panuveitis), keratitis, conjunctivitis, keratoconjunctivitis (including vernal keratoconjunctivitis (or “VKC”) and atopic keratoconjunctivitis), corneal ulcer, corneal edema, sterile corneal infiltrates, anterior scleritis, episcleritis, blepharitis, and post-operative (or post-surgical) ocular inflammation resulting from procedures such as photorefractive keratectomy, cataract removal surgery, intraocular lens (“IOL”) implantation, laser-assisted in situ keratomileusis (“LASIK”), conductive keratoplasty, radial keratotomy, dry eye, macular degeneration, macular edema, diabetic retinopathy, age-related macular degeneration (including the wet and dry forms), and glaucoma (including all types of glaucoma).

In still another aspect, such inflammatory diseases, conditions, or disorders result from an infection caused by bacteria, viruses, fungi, or protozoans.

In yet another aspect, the compositions comprise at least a mimetic of a glucocorticoid for treating or controlling such diseases, conditions, or disorders.

In yet another aspect, a pharmaceutical composition for treating or controlling an inflammatory disease, condition, or disorder comprises at least a dissociated glucocorticoid receptor agonist (“DIGRA”), a prodrug, or a pharmaceutically acceptable salt or ester thereof.

In yet another aspect, a pharmaceutical composition of the present invention comprises an ophthalmic topical formulation; injectable formulation; or implantable formulation, system, or device.

In still another aspect, such a formulation, system, or device is applied or provided to the anterior segment of the eye.

In still another aspect, such a formulation, system, or device is applied or provided to the posterior segment of the eye.

In a further aspect, said increased IOP is demonstrated in vitro or in vivo.

In yet another aspect, said increased IOP results from an increased resistance of fluid outflow through the trabecular meshwork.

In still another aspect, such increased resistance results from increased expression and accumulation of myocilin in the trabecular meshwork.

In another aspect, the present invention provides a method for treating or controlling an inflammatory disease, condition, or disorder of the anterior segment. The method comprises administering a composition comprising a DIGRA, a prodrug thereof, or a pharmaceutically acceptable salt or ester thereof to an affected eye of a subject in need of such treatment or control.

In still another aspect, said at least a DIGRA has Formula I.

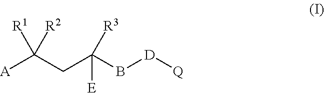

wherein A and Q are independently selected from the group consisting of unsubstituted and substituted aryl and heteroaryl groups, unsubstituted and substituted cycloalkyl and heterocycloalkyl groups, unsubstituted and substituted cycloalkenyl and heterocycloalkenyl groups, unsubstituted and substituted cycloalkynyl and heterocycloalkynyl groups, and unsubstituted and substituted heterocyclic groups; R¹ and R² are independently selected from the group consisting of hydrogen, unsubstituted C₁-C₁₅ (alternatively, C₁-C₁₀, or C₁-C₅, or C₁-C₃) linear or branched alkyl groups, substituted C₁-C₁₅ (alternatively, C₁-C₁₀, or C₁-C₅, or C₁-C₃) linear or branched alkyl groups, unsubstituted C₃-C₁₅ cycloalkyl groups, and substituted C₃-C₁₅ (alternatively, C₃-C₆, or C₃-C₅) cycloalkyl groups; R³ is selected from the group consisting of hydrogen, unsubstituted C₁-C₁₅ (alternatively, C₁-C₁₀, or C₁-C₅, or C₁-C₃) linear or branched alkyl groups, substituted C₁-C₁₅ (alternatively, C₁-C₁₀, or C₁-C₅, or C₁-C₃) linear or branched alkyl groups, unsubstituted C₃-C₁₅ (alternatively, C₃-C₆, or C₃-C₅) cycloalkyl and heterocycloalkyl groups, substituted C₃-C₁₅ (alternatively, C₃-C₆, or C₃-C₅) cycloalkyl and heterocycloalkyl groups, aryl groups, heteroaryl groups, and heterocyclylic groups; B comprises a carbonyl, amino, divalent hydrocarbon, or heterohydrocarbon group; E is hydroxy or amino group; and D is absent or comprises a carbonyl group, —NH—, or —NR′—, wherein R′ comprises an unsubstituted or substituted C₁-C₁₅ (alternatively, C₁-C₁₀), or C₁-C₅, or C₁-C₃) linear or branched alkyl group; and wherein R¹ and R² together may form an unsubstituted or substituted C₃-C₁₅ cycloalkyl group.

In one embodiment, B can comprise one or more unsaturated carbon-carbon bonds.

In another embodiment, B can comprise an alkylenecarbonyl, alkyleneoxycarbonyl, alkylenecarbonyloxy, alkyleneoxycarbonylamino, alkyleneamino, alkenylenecarbonyl, alkenyleneoxycarbonyl, alkenylenecarbonyloxy, alkenyleneoxycarbonylamino, alkenyleneamino, alkynylenecarbonyl, alkynyleneoxycarbonyl, alkynylenecarbonyloxy, alkynyleneoxycarbonylamino, alkynyleneamino, arylcarbonyloxy, aryloxycarbonyl, or ureido group.

In still another embodiment, A and Q are independently selected from the group consisting of aryl and heteroaryl groups substituted with at least a halogen atom, cyano group, hydroxy group, or C₁-C₁₀ alkoxy group (alternatively, C₁-C₅ alkoxy group, or C₁-C₃ alkoxy group); R¹, R², and R³ are independently selected from the group consisting of unsubstituted and substituted C₁-C₅ alkyl groups (preferably, C₁-C₃ alkyl groups); B is a C₁-C₅ alkylene group (alternatively, C₁-C₃ alkyl groups); D is the —NH— or —NR′— group, wherein R′ is a C₁-C₅ alkyl group (preferably, C₁-C₃ alkyl group); and E is the hydroxy group.

In yet another embodiment, A comprises a dihydrobenzofuranyl group substituted with a halogen atom; Q comprises a quinolinyl or isoquinolinyl group substituted with a C₁-C₁₀ alkyl group; R¹ and R² are independently selected from the group consisting of unsubstituted and substituted C₁-C₅ alkyl groups (preferably, C₁-C₃ alkyl groups); B is a C₁-C₃ alkylene group; D is the —NH— group; E is the hydroxy group; and R³ comprises a completely halogenated C₁-C₁₀ alkyl group (preferably, completely halogenated C₁-C₅ alkyl group; more preferably, completely halogenated C₁-C₃ alkyl group).

In still another embodiment, A comprises a dihydrobenzofuranyl group substituted with a fluorine atom; Q comprises a quinolinyl or isoquinolinyl group substituted with a methyl group; R¹ and R² are independently selected from the group consisting of unsubstituted and substituted C₁-C₅ alkyl groups; B is a C₁-C₃ alkylene group; D is the —NH— group; E is the hydroxy group; and R³ comprises a trifluoromethyl group.

In a further embodiment, said at least a DIGRA has Formula II or III.

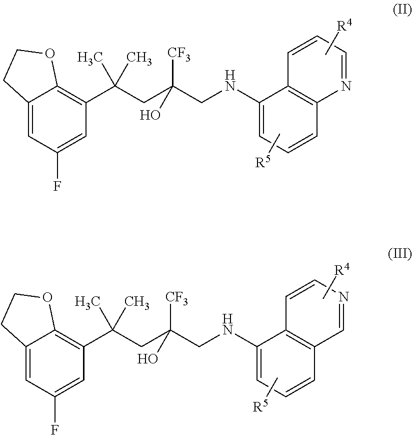

wherein R⁴ and R⁵ are independently selected from the group consisting of hydrogen, halogen, cyano, hydroxy, C₁-C₁₀ (alternatively, C₁-C₅ or C₁-C₃) alkoxy groups, unsubstituted C₁-C₁₀ (alternatively, C₁-C₅ or C₁-C₃) linear or branched alkyl groups, substituted C₁-C₁₀ (alternatively, C₁-C₅ or C₁-C₃) linear or branched alkyl groups, unsubstituted C₃-C₁₀ (alternatively, C₃-C₆ or C₃-C₅) cyclic alkyl groups, and substituted C₃-C₁₀ (alternatively, C₃-C₆ or C₃-C₅) cyclic alkyl groups.

In still another embodiment, said at least a DIGRA has Formula IV.

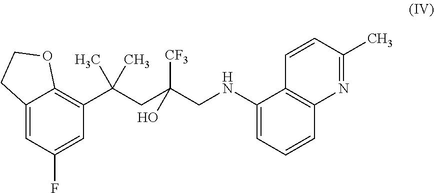

Methods for preparing compounds of Formula I, II, III, or IV are disclosed, for example, in U.S. Pat. Nos. 6,897,224; 6,903,215; 6,960,581, which are incorporated herein by reference in their entirety. Still other methods for preparing such compounds also can be found in U.S. Patent Application Publication 2006/0116396, which is incorporated herein by reference, or PCT Patent Application WO 2006/050998 A1.

Non-limiting examples of compounds having Formula I include 5-[4-(5-fluoro-2,3-dihydrobenzofuran-7-yl)-2-hydroxy-4-methyl-2-trifluoromethyl-pentylamino]-2-methylquinoline, 5-[4-(5-fluoro-2,3-dihydrobenzofuran-7-yl)-2-hydroxy-4-methyl-2-trifluoromethyl-pentylamino]-1-methylisoquinoline, 5-[4-(5-fluoro-2,3-dihydrobenzofuran-7-yl)-2-hydroxy-4-methyl-2-trifluoromethyl-pentylamino]isoquinol-1(2H)-one, 5-[4-(5-fluoro-2,3-dihydrobenzofuran-7-yl)-2-hydroxy-4-methyl-2-trifluoromethyl-pentylamino]-2,6-dimethylquinoline, 5-[4-(5-fluoro-2,3-dihydrobenzofuran-7-yl)-2-hydroxy-4-methyl-2-trifluoromethyl-pentylamino]-6-chloro-2-methylquinoline, 5-[4-(5-fluoro-2,3-dihydrobenzofuran-7-yl)-2-hydroxy-4-methyl-2-trifluoromethyl-pentylamino]isoquinoline, 5-[4-(5-fluoro-2,3-dihydrobenzofuran-7-yl)-2-hydroxy-4-methyl-2-trifluoromethyl-pentylamino]quinoline, 5-[4-(2,3-dihydro-5-fluoro-7-benzofuranyl)-2-hydroxy-4-methyl-2-trifluoromethyl-pentylamino]quinolin-2[1H]-one, 6-fluoro-5-[4-(5-fluoro-2,3-dihydrobenzofuran-7-yl)-2-hydroxy-4-methyl-2-trifluoromethyl-pentylamino]-2-methylquinoline, 8-fluoro-,5-[4-(5-fluoro-2,3-dihydrobenzofuran-7-yl)-2-hydroxy-4-methyl-2-trifluoromethyl-pentylamino]-2-methylquinoline, 5-[4-(5-fluoro-2,3-dihydrobenzofuran-7-yl)-2-hydroxy-4-methyl-2-trifluoromethyl-pentylamino]-2-methylisoquinol-1-[2h]-one, and enantiomers thereof.

Other compounds that can function as DIGRAs and methods for their manufacture are disclosed, for example, in U.S. Patent Application Publications 2004/0029932, 2004/0162321, 2004/0224992, 2005/0059714, 2005/0176706, 2005/0203128, 2005/0234091, 2005/0282881, 2006/0014787, 2006/0030561, 2006/0116396, 2006/0189646, and 2006/0189647, all of which are incorporated herein by reference in their entirety.

In another aspect, the present invention provides an ophthalmic pharmaceutical composition for treating or controlling an anterior-segment infection and its inflammatory sequalae. In one embodiment, such inflammatory sequalae comprise acute inflammation. In another embodiment, such inflammatory sequalae comprise chronic inflammation of the anterior segment. The ophthalmic pharmaceutical composition comprises a DIGRA, a prodrug thereof, or a pharmaceutically acceptable salt or ester thereof.

In another aspect, the composition further comprises an anti-infective agent.

In still another aspect, the pharmaceutical composition further comprises a pharmaceutically acceptable carrier.

The concentration of a DIGRA, a prodrug thereof, or a pharmaceutically acceptable salt or ester thereof in such an ophthalmic composition can be in the range from about 0.0001 to about 1000 mg/ml (or, alternatively, from about 0.001 to about 500 mg/ml, or from about 0.001 to about 300 mg/ml, or from about 0.001 to about 250 mg/ml, or from about 0.001 to about 100 mg/ml, or from about 0.001 to about 50 mg/ml, or from about 0.01 to about 300 mg/ml, or from about 0.01 to about 250 mg/ml, or from about 0.01 to about 100 mg/ml, or from about 0.1 to about 100 mg/ml, or from about 0.1 to about 50 mg/ml).

In one embodiment, a composition of the present invention is in a form of a suspension, dispersion, gel, or ointment. In another embodiment, the suspension or dispersion is based on an aqueous solution. For example, a composition of the present invention can comprise sterile saline solution. In still another embodiment, micrometer- or nanometer-sized particles of a DIGRA, or prodrug thereof, or a pharmaceutically acceptable salt or ester thereof and can be coated with a physiologically acceptable surfactant (non-limiting examples are disclosed below), then the coated particles are dispersed in a liquid medium. The coating can keep the particles in a suspension. Such a liquid medium can be selected to produce a sustained-release suspension. For example, the liquid medium can be one that is sparingly soluble in the ocular environment into which the suspension is administered.

An anti-infective agent suitable for a composition of the present invention is selected from the group consisting of antibacterial, antiviral, antifungal, antiprotozoal, and combinations thereof.

Non-limiting examples of biologically-derived antibacterial agents include aminoglycosides (e.g., amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin sv, rifapentine, rifaximin), β-lactams (e.g., carbacephems (e.g., loracarbef), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefinenoxime, cefodizime, cefonicid, cefoperazone, ceforanide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (e.g., cefbuperazone, cefinetazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, amoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin G benethamine, penicillin G benzathine, penicillin G benzhydrylamine, penicillin G calcium, penicillin G hydrabamine, penicillin G potassium, penicillin G procaine, penicillin N, penicillin O, penicillin V, penicillin V benzathine, penicillin V hydrabamine, penimepicycline, phenethicillin potassium, piperacillin, pivampicillin, propicillin, quinacillin, sulbenicillin, sultamicillin, talampicillin, temocillin, ticarcillin), ritipenem, lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin s, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), cycloserine, mupirocin, and tuberin.

Non-limiting examples of synthetic antibacterial agents include 2,4-diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, gatifloxacin, grepafloxacin, levofloxacin, lomefloxacin, miloxacin, moxifloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin, or a fluoroquinolone having the chemical name of 7-[(3R)-3-aminohexahydro-1H-azepin-1-yl]-8-chloro-1-cyclopropyl-6-fluoro-1,4-dihydro-4-oxo-3-quinolinecarboxylic acid monohydrochloride), sulfonamides (e.g., acetyl sulfamethoxypyrazine, benzylsulfamide, chloramines B, chloramines T, dichloramine T, n²-formylsulfisomidine, n⁴-β-D-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, 4-sulfanilamidosalicylic acid, n⁴-sulfanilylsulfanilamide, sulfanilylurea, N-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), clofoctol, hexedine, methenamine, methenamine anhydromethylene citrate, methenamine hippurate, methenamine mandelate, methenamine sulfosalicylate, nitroxoline, taurolidine, and xibomol. In one embodiment, a composition of the present invention comprises an anti-infective agent selected from the group consisting of cinoxacin, ciprofloxacin, clinafloxacin, difloxacin, enoxacin, fleroxacin, flumequine, gatifloxacin, grepafloxacin, levofloxacin, lomefloxacin, miloxacin, moxifloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin, and a fluoroquinolone having the chemical name of 7-[(3R)-3-aminohexahydro-1H-azepin-1-yl]-8-chloro-1-cyclopropyl-6-fluoro-1,4-dihydro-4-oxo-3-quinolinecarboxylic acid monohydrochloride (as a species of the family of compounds disclosed in U.S. Pat. Nos. 5,385,900 and 5,447,926, which are incorporated herein by reference).

Non-limiting examples of antiviral agents include Rifampin, Ribavirin, Pleconaryl, Cidofovir, Acyclovir, Pencyclovir, Gancyclovir, Valacyclovir, Famciclovir, Foscarnet, Vidarabine, Amantadine, Zanamivir, Oseltamivir, Resquimod, antiproteases, PEGylated interferon (Pegasys™), anti HIV proteases (e.g. lopinivir, saquinivir, amprenavir, HIV fusion inhibitors, nucleotide HIV RT inhibitors (e.g., AZT, Lamivudine, Abacavir), non-nucleotide HIV RT inhibitors, Doconosol, interferons, butylated hydroxytoluene (BHT), and Hypericin.

Non-limiting examples of biologically-derived antifungal agents include polyenes (e.g., amphotericin B, candicidin, dermostatin, filipin, fungichromin, hachimycin, hamycin, lucensomycin, mepartricin, natamycin, nystatin, pecilocin, perimycin), azaserine, griseofulvin, oligomycins, neomycin undecylenate, pyrrolnitrin, siccanin, tubercidin, and viridin.

Non-limiting examples of synthetic antifungal agents include allylamines (e.g., butenafine, naftifine, terbinafine), imidazoles (e.g., bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, tolnaftate), triazoles (e.g., fluconazole, itraconazole, saperconazole, terconazole), acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, and zinc propionate.

Non-limiting examples of antiprotozoal agents include polymycin B sulfate, bacitracin zinc, neomycine sulfate (e.g., Neosporin), imidazoles (e.g., clotrimazole, miconazole, ketoconazole), aromatic diamidines (e.g., propamidine isethionate, Brolene), polyhexamethylene biguanide (“PHMB”), chlorhexidine, pyrimethamine (Daraprim®), sulfadiazine, folinic acid (leucovorin), clindamycin, and trimethoprim-sulfamethoxazole.

In one aspect, the anti-infective agent is selected from the group consisting of bacitracin zinc, chloramphenicol, ciprofloxacin hydrochloride, erythromycin, gatifloxacin, gentamycin sulfate, levofloxacin, moxifloxacin, ofloxacin, sulfacetamide sodium, polymyxin B, tobramycin sulfate, trifluridine, vidarabine, acyclovir, valacyclovir, famcyclovir, foscarnet, ganciclovir, formivirsen, cidofovir, amphotericin B, natamycin, fluconazole, itraconazole, ketoconazole, miconazole, polymyxin B sulfate, neomycin sulfate, clotrimazole, propamidine isethionate, polyhexamethylene biguanide, chlorhexidine, pyrimethamine, sulfadiazine, folinic acid (leucovorin), clindamycin, trimethoprim-sulfamethoxazole, and combinations thereof.

The concentration of an anti-infective agent in such an ophthalmic composition can be in the range from about 0.0001 to about 1000 mg/ml (or, alternatively, from about 0.001 to about 500 mg/ml, or from about 0.001 to about 300 mg/ml, or from about 0.001 to about 250 mg/ml, or from about 0.001 to about 100 mg/ml, or from about 0.001 to about 50 mg/ml, or from about 0.01 to about 300 mg/ml, or from about 0.01 to about 250 mg/ml, or from about 0.01 to about 100 mg/ml, or from about 0.1 to about 100 mg/ml, or from about 0.1 to about 50 mg/ml).

In another aspect, a composition of the present invention can further comprise a non-ionic surfactant, such as polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Plutonic® F127 or Pluronic® F108)), or poloxamines (synthetic block polymers of ethylene oxide and propylene oxide attached to ethylene diamine, such as those commonly known by their trade names of Tetronic®; e.g., Tetronic® 1508 or Tetronic® 908, etc., other nonionic surfactants such as Brij®, Myrj®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). Such compounds are delineated in Martindale, 34^{th} ed., pp. 1411-1416 (Martindale, “The Complete Drug Reference,” S. C. Sweetman (Ed.), Pharmaceutical Press, London, 2005) and in Remington, “The Science and Practice of Pharmacy,” 21^{st} Ed., p. 291 and the contents of chapter 22, Lippincott Williams & Wilkins, New York, 2006); the contents of these sections are incorporated herein by reference. The concentration of a non-ionic surfactant, when present, in a composition of the present invention can be in the range from about 0.001 to about 5 weight percent (or alternatively, from about 0.01 to about 4, or from about 0.01 to about 2, or from about 0.01 to about 1, or from about 0.01 to about 0.5 weight percent).

In addition, a composition of the present invention can include additives such as buffers, diluents, carriers, adjuvants, or other excipients. Any pharmacologically acceptable buffer suitable for application to the eye may be used. Other agents may be employed in the composition for a variety of purposes. For example, buffering agents, preservatives, co-solvents, oils, humectants, emollients, stabilizers, or antioxidants may be employed. Water-soluble preservatives which may be employed include sodium bisulfite, sodium bisulfate, sodium thiosulfate, benzalkonium chloride, chlorobutanol, thimerosal, ethyl alcohol, methylparaben, polyvinyl alcohol, benzyl alcohol, and phenylethyl alcohol. These agents may be present in individual amounts of from about 0.001 to about 5% by weight (preferably, about 0.01% to about 2% by weight). Suitable water-soluble buffering agents that may be employed are sodium carbonate, sodium borate, sodium phosphate, sodium acetate, sodium bicarbonate, etc., as approved by the United States Food and Drug Administration (“US FDA”) for the desired route of administration. These agents may be present in amounts sufficient to maintain a pH of the system of between about 2 and about 11. As such, the buffering agent may be as much as about 5% on a weight to weight basis of the total composition. Electrolytes such as, but not limited to, sodium chloride and potassium chloride may also be included in the formulation.

In one aspect, the pH of the composition is in the range from about 4 to about 11. Alternatively, the pH of the composition is in the range from about 5 to about 9, from about 6 to about 9, or from about 6.5 to about 8. In another aspect, the composition comprises a buffer having a pH in one of said pH ranges.

In another aspect, the composition has a pH of about 7. Alternatively, the composition has a pH in a range from about 7 to about 7.5.

In still another aspect, the composition has a pH of about 7.4.

In yet another aspect, a composition also can comprise a viscosity-modifying compound designed to facilitate the administration of the composition into the subject or to promote the bioavailability in the subject. In still another aspect, the viscosity-modifying compound may be chosen so that the composition is not readily dispersed after being administered into the vistreous. Such compounds may enhance the viscosity of the composition, and include, but are not limited to: monomeric polyols, such as, glycerol, propylene glycol, ethylene glycol; polymeric polyols, such as, polyethylene glycol; various polymers of the cellulose family, such as hydroxypropylmethyl cellulose (“HPMC”), carboxymethyl cellulose (“CMC”) sodium, hydroxypropyl cellulose (“HPC”); polysaccharides, such as hyaluronic acid and its salts, chondroitin sulfate and its salts, dextrans, such as, dextran 70; water soluble proteins, such as gelatin; vinyl polymers, such as, polyvinyl alcohol, polyvinylpyrrolidone, povidone; carbomers, such as carbomer 934P, carbomer 941, carbomer 940, or carbomer 974P; and acrylic acid polymers. In general, a desired viscosity can be in the range from about 1 to about 400 centipoises (“cps”) or mPa·s.

In yet another aspect, the present invention provides a composition for treating or controlling an ophthalmic (anterior and or posterior segment) inflammatory disease, condition, or disorder. In one embodiment, the composition comprises: (a) at least a DIGRA, a prodrug thereof, or a pharmaceutically acceptable salt or ester thereof and (b) an anti-inflammatory agent other than said DIGRA, prodrug thereof, and pharmaceutically acceptable salt or ester thereof. In another embodiment, the anti-inflammatory agent is not a GC.

In still another aspect, such an anti-inflammatory agent comprises a compound that inhibits or blocks a cyclooxygenase inflammatory pathway, a lipoxygenase inflammatory pathway, or both.

In still another aspect, such an anti-inflammatory agent comprises a compound that inhibits or blocks production of a prostaglandin, thromboxane, or leukotriene.

In yet another aspect, the present invention provides a composition for treating or controlling an ophthalmic (anterior and/or posterior segment) inflammatory disease, condition, or disorder. In one embodiment, the composition comprises: (a) at least a DIGRA, a prodrug thereof, or a pharmaceutically acceptable salt or ester thereof (b) an anti-infective agent; and (c) an anti-inflammatory agent other than said DIGRA, prodrug thereof, and pharmaceutically acceptable salt or ester thereof. The DIGRA, anti-infective agent, and anti-inflammatory agent other than said DIGRA, prodrug thereof, and pharmaceutically acceptable salt or ester thereof are present in amounts effective to treat or control the disease, condition, or disorder. In one embodiment, such an anti-inflammatory agent is selected from the group consisting of non-steroidal anti-inflammatory drugs (“NSAIDs”); peroxisome proliferator-activated receptor (“PPAR”) ligands, such as PPARα, PPARδ, or PPARγ ligands; combinations thereof; and mixtures thereof.

Non-limiting examples of the NSAIDs are: aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), E-acetamidocaproic acid, S-(5′-adenosyl)-L-methionine, 3-amino-4-hydroxybutyric acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, their physiologically acceptable salts, combinations thereof, and mixtures thereof.

In certain embodiments, said anti-inflammatory agent other than said DIGRA, prodrug thereof, and pharmaceutically acceptable salt or ester thereof is selected from the group consisting of flurbiprofen, suprofen, bromfenac, diclofenac, indomethacin, ketorolac, salts thereof, and combinations thereof.

In another aspect of the present invention, an anti-inflammatory agent is a PPAR-binding molecule. In one embodiment, such a PPAR-binding molecule is a PPARα-, PPARδ-, or PPARγ-binding molecule. In another embodiment, such a PPAR-binding molecule is a PPARα, PPARδ, or PPARγ agonist. Such a PPAR ligand binds to and activates PPAR to modulate the expression of genes containing the appropriate peroxisome proliferator response element in its promoter region.

PPARγ agonists can inhibit the production of TNF-α and other inflammatory cytokines by human macrophages (C-Y. Jiang et al., Nature, Vol. 391, 82-86 (1998)) and T lymphocytes (A. E. Giorgini et al., Horm. Metab. Res. Vol. 31, 1-4 (1999)). More recently, the natural PPARγ agonist 15-deoxy-A-12,14-prostaglandin J2 (or “15-deoxy-Δ-12,14-PG J2”), has been shown to inhibit neovascularization and angiogenesis (X. Xin et al., J. Biol. Chem. Vol. 274:9116-9121 (1999)) in the rat cornea. Spiegelman et al., in U.S. Pat. No. 6,242,196, disclose methods for inhibiting proliferation of PPARγ-responsive hyperproliferative cells by using PPARγ agonists; numerous synthetic PPARγ agonists are disclosed by Spiegelman et al., as well as methods for diagnosing PPARγ-responsive hyperproliferative cells. All documents referred to herein are incorporated by reference. PPARs are differentially expressed in diseased versus normal cells. PPARγ is expressed to different degrees in the various tissues of the eye, such as some layers of the retina and the cornea, the choriocapillaris, uveal tract, conjunctival epidermis, and intraocular muscles (see, e.g., U.S. Pat. No. 6,316,465).

In one aspect, a PPARγ agonist used in a composition or a method of the present invention is a thiazolidinedione, a derivative thereof, or an analog thereof. Non-limiting examples of thiazolidinedione-based PPARγ agonists include pioglitazone, troglitazone, ciglitazone, englitazone, rosiglitazone, and chemical derivatives thereof. Other PPARγ agonists include Clofibrate (ethyl 2-(4-chlorophenoxy)-2-methylpropionate), clofibric acid (2-(4-chlorophenoxy)-2-methylpropanoic acid), GW 1929 (N-(2-benzoylphenyl)-O-{2-(methyl-2-pyridinylamino)ethyl}-L-tyrosine), GW 7647 (2-{{4-{2-{{(cyclohexylamino)carbonyl}(4-cyclohexylbutyl)amino}ethyl}phenyl}thio}-2-methylpropanoic acid), and WY 14643 ({{4-chloro-6-{(2,3-dimethylphenyl)amino}-2-pyrimidinyl}thio}acetic acid). GW 1929, GW 7647, and WY 14643 are commercially available, for example, from Koma Biotechnology, Inc. (Seoul, Korea). In one embodiment, the PPARγ agonist is 15-deoxy-Δ-12, 14-PG J2.

Non-limiting examples of PPAR-α agonists include the fibrates, such as fenofibrate and gemfibrozil. A non-limiting example of PPAR-δ agonist is GW501516 (available from Axxora LLC, San Diego, Calif. or EMD Biosciences, Inc., San Diego, Calif.).

The concentration of any foregoing additional active ingredient in such an ophthalmic composition can be in the range from about 0.0001 to about 1000 mg/ml (or, alternatively, from about 0.001 to about 500 mg/ml, or from about 0.001 to about 300 mg/ml, or from about 0.001 to about 250 mg/ml, or from about 0.001 to about 100 mg/ml, or from about 0.001 to about 50 mg/ml, or from about 0.01 to about 300 mg/ml, or from about 0.01 to about 250 mg/ml, or from about 0.01 to about 100 mg/ml, or from about 0.1 to about 100 mg/ml, or from about 0.1 to about 50 mg/ml).

In still another aspect, a method for preparing a composition of the present invention comprises combining: (a) at least a DIGRA, a prodrug thereof, or a pharmaceutically acceptable salt or ester thereof (b) a pharmaceutically acceptable carrier; and (c) a material selected from the group consisting of (i) an anti-infective agent, (ii) an anti-inflammatory agent other than said DIGRA, prodrug thereof, and pharmaceutically acceptable salt or ester thereof; and (iii) combinations thereof. In one embodiment, such a carrier can be a sterile saline solution or a physiologically acceptable buffer. In another embodiment, such a carrier comprises a hydrophobic medium, such as a pharmaceutically acceptable oil. In still another embodiment, such as carrier comprises an emulsion of a hydrophobic material and water.

Physiologically acceptable buffers include, but are not limited to, a phosphate buffer or a Tris-HCl buffer (comprising tris(hydroxymethyl)aminomethane and HCl). For example, a Tris-HCl buffer having pH of 7.4 comprises 3 g/l of tris(hydroxymethyl)aminomethane and 0.76 g/l of HCl. In yet another aspect, the buffer is 10× phosphate buffer saline (“PBS”) or 5×PBS solution.

Other buffers also may be found suitable or desirable in some circumstances, such as buffers based on HEPES (N-{2-hydroxyethyl}peperazine-N′-{2-ethanesulfonic acid}) having pKₐ of 7.5 at 25° C. and pH in the range of about 6.8-8.2; BES (N,N-bis{2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; TES (N-tris{hydroxymethyl}-methyl-2-aminoethanesulfonic acid) having pKₐ of 7.4 at 25° C. and pH in the range of about 6.8-8.2; MOBS (4-{N-morpholino}butanesulfonic acid) having pKₐ of 7.6 at 25° C. and pH in the range of about 6.9-8.3; DIPSO (3-(N,N-bis{2-hydroxyethyl}amino)-2-hydroxypropane)) having pKₐ of 7.52 at 25° C. and pH in the range of about 7-8.2; TAPSO (2-hydroxy-3{tris(hydroxymethyl)methylamino}-1-propanesulfonic acid)) having pKₐ of 7.61 at 25° C. and pH in the range of about 7-8.2; TAPS ({(2-hydroxy-1,1-bis(hydroxymethyl)ethyl)amino}-1-propanesulfonic acid)) having pKₐ of 8.4 at 25° C. and pH in the range of about 7.7-9.1; TABS (N-tris(hydroxymethyl)methyl-4-aminobutanesulfonic acid) having pKₐ of 8.9 at 25° C. and pH in the range of about 8.2-9.6; AMPSO (N-(1,1-dimethyl-2-hydroxyethyl)-3-amino-2-hydroxypropanesulfonic acid)) having pKₐ of 9.0 at 25° C. and pH in the range of about 8.3-9.7; CHES (2-cyclohexylamino)ethanesulfonic acid) having pKₐ of 9.5 at 25° C. and pH in the range of about 8.6-10.0; CAPSO (3-(cyclohexylamino)-2-hydroxy-1-propanesulfonic acid) having pKₐ of 9.6 at 25° C. and pH in the range of about 8.9-10.3; or CAPS (3-(cyclohexylamino)-1-propane sulfonic acid) having pKₐ of 10.4 at 25° C. and pH in the range of about 9.7-11.1.

In certain embodiments, a composition of the present invention is formulated in a buffer having an acidic pH, such as from about 4 to about 6.8, or alternatively, from about 5 to about 6.8. In such embodiments, the buffer capacity of the composition desirably allows the composition to come rapidly to a physiological pH after being administered into the patient.

It should be understood that the proportions of the various components or mixtures in the following examples may be adjusted for the appropriate circumstances.

### Example 1

Two mixtures I and II are made separately by mixing the ingredients listed in Table 1. Five parts (by weight) of mixture I are mixed with twenty parts (by weight) of mixture II for 15 minutes or more. The pH of the combined mixture is adjusted to 6.2-6.4 using 1 N NaOH or 1 N HCl solution to yield a composition of the present invention.

TABLE 1

| | |
|---|---|
| Ingredient | Amount |

Mixture I

| | | |
|---|---|---|
| Carbopol 934P NF | 0.25 | g |
| Purified water | 99.75 | g |

Mixture II

| | | |
|---|---|---|
| Propylene glycol | 5 | g |
| EDTA | 0.1 | mg |
| Compound of Formula IV | 50 | g |

### Example 2

Two mixtures I and II are made separately by mixing the ingredients listed in Table 2. Five parts (by weight) of mixture I are mixed with twenty parts (by weight) of mixture II for 15 minutes or more. The pH of the combined mixture is adjusted to 6.2-6.4 using 1 N NaOH or 1 N HCl solution to yield a composition of the present invention.

TABLE 2

| | |
|---|---|
| Ingredient | Amount |

Mixture I

| | | |
|---|---|---|
| moxifloxacin | 0.2 | g |
| diclofenac | 0.3 | g |
| Carbopol 934P NF | 0.25 | g |
| Purified water | 99.25 | g |

Mixture II

| | | |
|---|---|---|
| Propylene glycol | 5 | g |
| EDTA | 0.1 | mg |
| Compound of Formula IV | 50 | g |

### Example 3

Two mixtures I and II are made separately by mixing the ingredients listed in Table 3. Five parts (by weight) of mixture I are mixed with twenty parts (by weight) of mixture II for 15 minutes or more. The pH of the combined mixture is adjusted to 6.2-6.4 using 1 N NaOH or 1 N HCl solution to yield a composition of the present invention.

TABLE 3

| | |
|---|---|
| Ingredient | Amount |

Mixture I

| | | |
|---|---|---|
| gatifloxacin | 0.2 | g |
| ciglitazone | 0.2 | g |
| Carbopol 934P NF | 0.25 | g |
| Purified water | 99.35 | g |

Mixture II

| | | |
|---|---|---|
| Propylene glycol | 3 | g |
| Triacetin | 7 | g |
| Compound of Formula II | 50 | g |
| EDTA | 0.1 | mg |

### Example 4

Two mixtures I and II are made separately by mixing the ingredients listed in Table 4. Five parts (by weight) of mixture I are mixed with twenty parts (by weight) of mixture II for 15 minutes or more. The pH of the combined mixture is adjusted to 6.2-7.5 using 1 N NaOH or 1 N HCl solution to yield a composition of the present invention.

TABLE 4

| | |
|---|---|
| Ingredient | Amount |

Mixture I

| | | |
|---|---|---|
| tobramycin sulfate | 0.3 | g |
| gemfibrozil | 0.3 | g |
| Carbopol 934P NF | 0.25 | g |
| Olive oil | 99.15 | g |

Mixture II

| | | |
|---|---|---|
| Propylene glycol | 7 | g |
| Glycerin | 3 | g |
| Compound of Formula III | 50 | g |
| Cyclosporine A | 5 | g |
| HAP (30%) | 0.5 | mg |
| Alexidine 2HCl | 1-2 | ppm |

Note:
“HAP” denotes hydroxyalkyl phosphonates, such as those known under the trade name Dequest ®.

### Example 5

The ingredients listed in Table 5 are mixed together for at least 15 minutes. The pH of the mixture is adjusted to 6.2-7.5 using 1 N NaOH or 1 N HCl solution to yield a composition of the present invention.

TABLE 5

| Ingredient | Amount (% by weight) |
|---|---|
| Povidone | 1 |
| HAP (30%) | 0.05 |
| Glycerin | 3 |
| Propylene glycol | 3 |
| Compound of Formula IV | 0.5 |
| Tyloxapol | 0.25 |
| BAK | 10-100 ppm |
| Purified water | q.s. to 100 |

Note:
“BAK” denotes benzalkonium chloride.

### Example 6

The ingredients listed in Table 6 are mixed together for at least 15 minutes. The pH of the mixture is adjusted to 7-7.5 using 1 N NaOH or 1 N HCl solution to yield a composition of the present invention.

TABLE 6

| Ingredient | Amount (% by weight) |
|---|---|
| Povidone | 1.5 |
| HAP (30%) | 0.05 |
| Glycerin | 3 |
| Propylene glycol | 3 |
| Compound of Formula III | 0.75 |
| Tyloxapol | 0.25 |
| Alexidine 2HCl | 1-2 ppm |
| Purified water | q.s. to 100 |

### Example 7

The ingredients listed in Table 7 are mixed together for at least 15 minutes. The pH of the mixture is adjusted to 6.5-7.8 using 1 N NaOH or 1 N HCl solution to yield a composition of the present invention.

TABLE 7

| Ingredient | Amount (% by weight) |
|---|---|
| CMC (MV) | 0.5 |
| HAP (30%) | 0.05 |
| Glycerin | 3 |
| Propylene glycol | 3 |
| Compound of Formula II | 0.75 |
| Tyloxapol (a surfactant) | 0.25 |
| Alexidine 2HCl | 1-2 ppm |
| Purified water | q.s. to 100 |

### Example 8

The ingredients listed in Table 8 are mixed together for at least 15 minutes. The pH of the mixture is adjusted to 6.2-7.4 using 1 N NaOH or 1 N HCl solution to yield a composition of the present invention.

TABLE 8

| Ingredient | Amount (% by weight) |
|---|---|
| CMC (MV) | 0.5 |
| HAP (30%) | 0.05 |
| Glycerin | 3 |
| Propylene glycol | 3 |
| Compound of Formula IV | 0.75 |
| Miconazole | 0.2 |
| 15-deoxy-Δ-12,14-prostaglandin J2 | 0.3 |
| Tyloxapol (a surfactant) | 0.25 |
| Alexidine 2HCl | 1-2 ppm |
| Purified water | q.s. to 100 |

### Example 9

The ingredients listed in Table 9 are mixed together for at least 15 minutes. The pH of the mixture is adjusted to 6.2-6.8 using 1 N NaOH or 1 N HCl solution to yield a composition of the present invention.

TABLE 9

| Ingredient | Amount (% by weight) |
|---|---|
| CMC (MV) | 0.5 |
| HAP (30%) | 0.05 |
| Glycerin | 3 |
| Propylene glycol | 3 |
| Compound of Formula IV | 0.75 |
| Bacitracin zinc | 0.2 |
| Flurbiprofen | 0.2 |
| Levofloxacin | 0.3 |
| Tyloxapol (a surfactant) | 0.25 |
| Alexidine 2HCl | 1-2 ppm |
| Purified water | q.s. to 100 |

### Example 10

The ingredients listed in Table 10 are mixed together for at least 15 minutes. The pH of the mixture is adjusted to 6.2-6.8 using 1 N NaOH or 1 N HCl solution to yield a composition of the present invention.

TABLE 10

| Ingredient | Amount (% by weight) |
|---|---|
| CMC (MV) | 0.5 |
| HAP (30%) | 0.05 |
| Glycerin | 3 |
| Propylene glycol | 3 |
| Compound of Formula III | 0.75 |
| Moxifloxacin | 0.2 |
| 15-deoxy-Δ-12,14-prostaglandin J2 | 0.3 |
| clotrimazole | 0.2 |
| Tyloxapol (a surfactant) | 0.25 |
| Alexidine 2HCl | 1-2 ppm |
| Purified water | q.s. to 100 |

### Example 11

The ingredients listed in Table 11 are mixed together for at least 15 minutes. The pH of the mixture is adjusted to 6.2-7 using 1 N NaOH or 1 N HCl solution to yield a composition of the present invention.

TABLE 11

| Ingredient | Amount | |
|---|---|---|
| Ketorolac | 0.4 | g |
| Compound having Formula IV | 0.2 | g |
| Carbopol 934P NF | 0.25 | g |
| Propylene glycol | 5 | g |
| EDTA | 0.5 | mg |
| Purified water | 98.65 | g |

In another aspect, a DIGRA, a prodrug thereof, or a pharmaceutically acceptable salt or ester thereof is incorporated into a formulation for topical administration or periocular injection to a portion of the anterior segment. An injectable formulation can desirably comprise a carrier that provides a sustained-release of the active ingredients, such as for a period longer than about 1 week (or longer than about 1, 2, 3, 4, 5, or 6 months). In certain embodiments, the sustained-release formulation desirably comprises a carrier that is insoluble or only sparingly soluble in the anterior-segment environment. Such a carrier can be an oil-based liquid, emulsion, gel, or semisolid. Non-limiting examples of oil-based liquids include castor oil, peanut oil, olive oil, coconut oil, sesame oil, cottonseed oil, corn oil, sunflower oil, fish-liver oil, arachis oil, and liquid paraffin.

In another embodiment, the formulation further comprises a material selected from the group consisting of: (i) anti-infective agents; (ii) anti-inflammatory agents other than said DIGRA, prodrug thereof, pharmaceutically acceptable salts, and pharmaceutically acceptable esters thereof; and (iii) a combination thereof.

In one embodiment, a compound or composition of the present invention can be injected with a fine-gauge needle, such as 25-35 gauge. Typically, an amount from about 25 μl to about 100 μl of a composition comprising a DIGRA, a prodrug thereof, or a pharmaceutically acceptable salt or ester thereof is administered into a patient. A concentration of such DIGRA, prodrug thereof, or pharmaceutically acceptable salt or ester thereof is selected from the ranges disclosed above.

In still another aspect, a DIGRA, a prodrug thereof, or a pharmaceutically acceptable salt or ester thereof is incorporated into an ophthalmic device that comprises a biodegradable material, and the device is implanted into an anterior-segment tissue of a subject to provide a long-term (e.g., longer than about 1 week, or longer than about 1, 2, 3, 4, 5, or 6 months) treatment or control of an anterior-segment inflammatory disease, condition, or disorder. Such a device may be implanted by a skilled physician in the subject's ocular or periocular tissue.

In still another aspect, a method for treating or controlling an ocular inflammatory disease, condition, or disorder comprises: (a) providing a composition comprising a DIGRA, a prodrug thereof, or a pharmaceutically acceptable salt or ester thereof; and (b) administering to a subject an amount of the composition at a frequency sufficient to treat or control said ocular disease, condition, or disorder in a subject, wherein said method results in a lower risk of increased IOP in said subject than a method using a prior-art GC. In one embodiment, said prior-art GC is dexamethasone. In another embodiment, said prior-art GC is prednisolone acetate.

In still another aspect, a method for treating or controlling a post-operative inflammation of the anterior segment comprises: (a) providing a composition comprising a DIGRA, a prodrug thereof, or a pharmaceutically acceptable salt or ester thereof; and (b) administering to a subject an amount of the composition at a frequency sufficient to treat or control said post-operative inflammation, wherein said method results in a lower risk of increased IOP in said subject than a method using a prior-art GC. In one embodiment, said prior-art GC is dexamethasone. In another embodiment, said prior-art GC is prednisolone acetate.

In still another aspect, a method for treating or controlling an anterior-segment inflammatory disease, condition, or disorder comprises: (a) providing a composition comprising a DIGRA, a prodrug thereof, or a pharmaceutically acceptable salt or ester thereof; and (b) administering to a subject an amount of the composition at a frequency sufficient to treat or control an anterior-segment disease, condition, or disorder in a subject, wherein said method results in a lower risk of increased IOP in said subject than a method using a prior-art GC. In one embodiment, said prior-art GC is dexamethasone. In another embodiment, said prior-art GC is prednisolone acetate.

In still another aspect, a method for treating or controlling a post-operative inflammation of the anterior segment comprises: (a) providing a composition comprising a DIGRA, a prodrug thereof, or a pharmaceutically acceptable salt or ester thereof; and (b) administering to a subject an amount of the composition at a frequency sufficient to treat or control said post-operative inflammation, wherein said method results in a lower risk of increased IOP in said subject than a method using a prior-art GC. In one embodiment, said prior-art GC is dexamethasone. In another embodiment, said prior-art GC is prednisolone acetate.

In still another aspect, a method for treating or controlling an anterior-segment inflammatory disease, condition, or disorder comprises: (a) providing a composition comprising: (i) a DIGRA, a prodrug thereof, or a pharmaceutically acceptable salt or ester thereof; (ii) an anti-inflammatory agent other than said DIGRA, prodrug thereof, and pharmaceutically acceptable salt or ester thereof; and (iii) an anti-infective agent; and (b) administering to a subject an amount of the composition at a frequency sufficient to treat or control an anterior-segment disease, condition, or disorder in a subject, wherein said method results in a lower risk of increased IOP in said subject than a method using a prior-art GC. In one embodiment, said prior-art GC is dexamethasone. In another embodiment, said prior-art GC is prednisolone acetate.

In still another aspect, a method for treating or controlling a post-operative inflammation of the anterior segment comprises: (a) providing a composition comprising: (i) a DIGRA, a prodrug thereof, or a pharmaceutically acceptable salt or ester thereof; (ii) an anti-inflammatory agent other than said DIGRA, prodrug thereof, and pharmaceutically acceptable salt or ester thereof; and (iii) an anti-infective agent; and (b) administering to a subject an amount of the composition at a frequency sufficient to treat or control said post-operative inflammation, wherein said method results in a lower risk of increased IOP in said subject than a method using a prior-art GC. In one embodiment, said prior-art GC is dexamethasone. In another embodiment, said prior-art GC is prednisolone acetate.

In certain embodiments, the DIGRA is selected from among those disclosed above.

In other embodiments, the anti-inflammatory agent is selected from among those disclosed above. In some embodiments, the anti-inflammatory agent is selected from the group consisting of flurbiprofen, suprofen, bromfenac, diclofenac, indomethacin, ketorolac, salts thereof, and combinations thereof.

In another embodiment, such inflammation is a long-term inflammation. In still another embodiment, such inflammation requires at least two weeks for resolution, if untreated.

In still another embodiment, such inflammatory anterior-segment disease, condition, or disorder results from ophthalmic infection that is caused by a virus, bacteria, fungus, or protozoa.

In another aspect, a composition of the present invention is administered periocularly or in the anterior chamber. In still another aspect, a composition of the present invention is incorporated into an ophthalmic implant system or device, and the implant system or device is surgically implanted periocularly or in a tissue adjacent to the anterior portion of the eye of the patient for the sustained release of the active ingredient or ingredients. A typical implant system or device suitable for use in a method of the present invention comprises a biodegradable matrix with the active ingredient or ingredients impregnated or dispersed therein. Non-limiting examples of ophthalmic implant systems or devices for the sustained-release of an active ingredient are disclosed in U.S. Pat. Nos. 5,378,475; 5,773,019; 5,902,598; 6,001,386; 6,051,576; and 6,726,918; which are incorporated herein by reference.

In yet another aspect, a composition of the present invention is administered once a week, once a month, once a year, twice a year, four times a year, or at a suitable frequency that is determined to be appropriate for treating or controlling an anterior-segment inflammatory disease, condition, or disorder.

Comparison of Glucocorticoids and DIGRAS

One of the most frequent undesirable actions of a glucocorticoid therapy is steroid diabetes. The reason for this undesirable condition is the stimulation of gluconeogenesis in the liver by the induction of the transcription of hepatic enzymes involved in gluconeogenesis and metabolism of free amino acids that are produced from the degradation of proteins (catabolic action of glucocorticoids). A key enzyme of the catabolic metabolism in the liver is the tyrosine aminotransferase (“TAT”). The activity of this enzyme can be determined photometrically from cell cultures of treated rat hepatoma cells. Thus, the gluconeogenesis by a glucocorticoid can be compared to that of a DIGRA by measuring the activity of this enzyme. For example, in one procedure, the cells are treated for 24 hours with the test substance (a DIGRA or glucocorticoid), and then the TAT activity is measured. The TAT activities for the selected DIGRA and glucocorticoid are then compared. Other hepatic enzymes can be used in place of TAT, such as phosphoenolpyruvate carboxykinase, glucose-6-phosphatase, or fructose-2,6-biphosphatase. Alternatively, the levels of blood glucose in an animal model may be measured directly and compared for individual subjects that are treated with a glucocorticoid for a selected condition and those that are treated with a DIGRA for the same condition.

Another undesirable result of glucocorticoid therapy is GC-induced cataract. The cataractogenic potential of a compound or composition may be determined by quantifying the effect of the compound or composition on the flux of potassium ions through the membrane of lens cells (such as mammalian lens epithelial cells) in vitro. Such an ion flux may be determined by, for example, electrophysiological techniques or ion-flux imaging techniques (such as with the use of fluorescent dyes). An exemplary in-vitro method for determining the cataractogenic potential of a compound or composition is disclosed in U.S. Patent Application Publication 2004/0219512, which is incorporated herein by reference.

Still another undesirable result of glucocorticoid therapy is hypertension. Blood pressure of similarly matched subjects treated with glucocorticoid and DIGRA for an inflammatory condition may be measured directly and compared.

Yet another undesirable result of glucocorticoid therapy is increased IOP in the subject. IOP of similarly matched subjects treated with glucocorticoid and DIGRA for a condition may be measured directly and compared.

### TESTING: Comparison of the DIGRA Having Formula IV With Two Corticosteroids and One NSA/D in Treating Anterior-Segment Inflammation

### 1. Introduction

Inflammatory processes are multidimensional in origin, and are characterized by complex cellular and molecular events involving numerous components all of which have not been identified. Prostaglandins are among these mediators and play an important role in certain forms of ocular inflammation. Paracentesis of the anterior chamber in the rabbit eye induces inflammatory reaction due to the disruption of the blood-aqueous barrier (“BAB”), which is mediated, at least in part, by prostaglandin E₂ [References 1-3 below]. Intraocular or topical administration of PGE₂ disrupts the BAB. [Reference 4, below] The treatment schedule adopted in this study was similar to the clinical NSAIDs (Ocufen) treatment schedule used by surgeons for patients before cataract surgery. We investigated a dissociated glucocorticoid receptor agonist (“BOL-303242-X”, compound having Formula IV above) at different doses on rabbit paracentesis model evaluating aqueous biomarkers levels, and iris-ciliary body MPO activity in comparison with vehicle, dexamethasone, loteprednol and flurbiprofen.

### 2. Methods

### 2.1 Drugs and Materials

### 2.1.1. Test Articles

BOL-303242-X (0.1%, 0.5% and 1% topical formulations), lot 2676-MLC-107, Bausch & Lomb Incorporated (“B&L”) Rochester, USA.

Vehicle (10% PEG 3350; 1% Tween 80; phosphate buffer pH 7.00), lot 2676-MLC-107, B&L Rochester, USA.

Visumetazone® (0.1% Dexamethasone topical formulation), lot T253, Visufarma, Rome, Italy.

Lotemax (0.5% Loteprednol topical formulation), lot 078061, B&L IOM, Macherio, Italy.

Ocufen® (0.03% Flurbiprofen topical formulation), lot E45324, Allergan, Westport, Ireland.

### 2.2 Animals

Species: Rabbit

Breed: New Zealand

Source: Morini (Reggio Emila, Italy)

Sex: Male

Age at Experimental Start: 10 weeks.

Weight Range at Experimental Start: 2.0-2.4 Kg

Total Number of Animals: 28

Identification: Ear tagged with an alphanumeric code (i.e. A1 means test article A and animal 1).

Justification: The rabbit is a standard non-rodent species used in pharmacodynamic studies. The number of animals used in this study is, in judgment of the investigators involved, the minimum number necessary to properly perform this type of study and it is consistent with world wide regulatory guidelines.

Acclimation/Quarantine: Following arrival, a member of the veterinary staff assessed animals as to their general health. Seven days elapsed between animal receipt and the start of experiment in order to acclimate animals to the laboratory environment and to observe them for the development of infection disease.

Animal Husbandry: All the animals were housed in a cleaned and disinfected room, with a constant temperature (22±1° C.), humidity (relative, 30%) and under a constant light-dark cycle (light on between 8.00 and 20.00). Commercial food and tap water were available ad libitum. Their body weights were measured just before the experiment (Table T-1). All the animals had a body weight inside the central part of the body weight distribution curve (10%). Four rabbits were replaced with animals of similar age and weight from the same vendor because three of them showed signs of ocular inflammation and one was dead upon arrival.

Animals Welfare Provisions: All experiments were carried out according to the ARVO (Association for Research in Vision and Ophthalmology) guidelines on the use of animals in research. No alternative test system exists which have been adequately validated to permit replacement of the use of live animals in this study. Every effort has been made to obtain the maximum amount of information while reducing to a minimum the number of animals required for this study. To the best of our knowledge, this study is not unnecessary or duplicative. The study protocol was reviewed and approved by the Institutional Animal Care and Use Committee (IACUC) of the University of Catania and complies with the acceptable standards of animal welfare care.

### 2.3 Experimental Preparations

### 2.3.1 Study Design and Randomization

Twenty-eight rabbits were randomly allocated into 7 groups (4 animals/each) as shown in the table below.

TABLE 8

| | 2. No of | | | 4. Observations | 5. Termination |
|---|---|---|---|---|---|
| 1. Group | rabbits | 3. Treatment | | and measurements | and assays |
| 6. | 7. [symbol] | 8. CTR | 9. 50 μl | 10. Clinical | 13. Termination |
| 18. I | 19. [symbol] | 20. 1% | drops at 180, | observations and | immediately after |
| | | BOL | 120, 90, and | pupillary diameter at | the second |
| 21. | 22. [symbol] | 23. 0.5% | 30 min prior | 180 and 5 min before | paracentesis. |
| II | | BOL | to first | the first paracentesis, | 14. |
| 24. V | 25. [symbol] | 26. 0.1% | paracentesis, | and at 5 min before | 15. Aqueous |
| | | BOL | and at 15, 30, | the second | humor collected for |
| 27. | 28. [symbol] | 29. 0.5% | 90 min after | paracentesis. | PGE₂, protein, |
| | | LE | the first | 11. | leukocytes and |
| 30. I | 31. [symbol] | 32. 0.1% | paracentesis. | 12. Paracentesis | LTB₄ |
| | | Dex | | at 0 and 2 hours. | measurements. |
| 33. | 34. [symbol] | 35. 0.03% F | | | 16. |
| II | | | | | 17. Iris-ciliary |
| | | | | | body collected for |
| | | | | | MPO activity |
| | | | | | measurement. |

CTR = vehicle;
BOL = BOL-303242-X;
LE = loteprednol etabonate;
Dex = dexamethasone;
F = flurbiprofen [symbol]
indicates data missing or illegible when filed

To each test article was randomly assigned a letter from A to G

A=vehicle (10% PEG3350/1% Tween 80/PB pH 7.00)

B=Ocufen (Flurbiprofen 0.03%)

C=Visumetazone (Dexamethasone 0.1%)

D=Lotemax (Loteprednol etabonate 0.5%)

E=BOL-303242-X 0.1% (1 mg/g)

F=BOL-303242-X 0.5% (5 mg/g)

G=BOL-303242-X1% (10 mg/g)

### 2.3.2 Reagent Preparation for MPO Assay

2.3.2.1 Phosphate Buffer (50 mM; pH=6)

3.9 g of NaH₂PO₄ 2H₂O were dissolved in a volumetric flask to 500 ml with water. The pH was adjusted to pH=6 with 3N NaOH.

2.3.2.2 Hexa-decyl-trimethyl-ammonium bromide (0.5%)

0.5 g of hexa-decyl-trimethyl-ammonium bromide was dissolved in 100 ml phosphate buffer.

2.3.2.3 o-dianisidine 2HCl (0.0167%)/H₂O₂ (0.0005%) Solution

The solution was prepared freshly. Ten microliters of H₂O₂ (30 wt. %) were diluted to 1 ml with water (solution A). 7.5 mg o-dianisidine 2HCl was dissolved in 45 ml of phosphate buffer and 75 μl of solution A were added.

### 2.4 Experimental Protocols

### 2.4.1 Animals Treatment and Sample Collection

Each rabbit was placed in a restraint device and tagged with the alphanumeric code. The formulations were instilled (50 μl) into the conjunctival sac of both eyes 180, 120, 90 and 30 min before the first paracentesis; then 15, 30, 90 min after the first paracentesis. To perform the first paracentesis the animals were anaesthetized by intravenous injection of 5 mg/kg Zoletil® (Virbac; 2.5 mg/kg tiletamine HCl and 2.5 mg/kg zolazepam HCl) and one drop of local anesthetic (Novesina®, Novartis) was administered to the eye. Anterior chamber paracentesis was performed with a 26 G needle attached to a tuberculin syringe; the needle was introduced into the anterior chamber through the cornea, taking care not to damage the tissues. Two hours after the first paracentesis, the animals were sacrificed with 0.4 ml Tanax® (Intervet International B.V.) and the second paracentesis was performed. About 100 μl of aqueous humor were removed at the second paracentesis. Aqueous humor was immediately split in four aliquots and stored at −80° C. until analysis. Then both eyes were enucleated and the iris-ciliary body was carefully excised, placed in polypropylene tubes, and stored at −80° C. until analysis.

### 2.4.2 Pupillary Diameter Measurement

The pupillary diameter of both eyes was measured with a Castroviejo caliper 180 min and 5 min before the first paracentesis and 5 min before the second paracentesis.

### 2.4.3 Clinical Evaluation

The clinical evaluation of both eyes was performed by a slit lamp (4179-T; Sbisà, Italy) at 180 min and 5 min before the first paracentesis and 5 min before the second paracentesis. The clinical score was assigned according to the following scheme:

0=normal

1=discrete dilatation of iris and conjunctival vessels

2=moderate dilatation of iris and conjunctival vessels

3=intense iridal hyperemia with flare in the anterior chamber

4=intense iridal hyperemia with flare in the anterior chamber and presence of fibrinous exudates.

### 2.4.4 Prostaglandin E₂ (PGE₂) Measurement

For the quantitative determination of PGE₂ in the aqueous humor we used the PGE₂ Immunoassay kit (R&D Systems; Cat. No. KGE004; Lot. No. 240010). Eleven microliters or 16 μl of aqueous humor were diluted to 110 μl or 160 μl with the calibrator diluent solution provided with the kit. One hundred microliters of samples and of standards were load into a 96-well plate and recorded in a plate layout. Samples were treated following the assay procedure described in the kit. A microplate reader (GDV, Italy; model DV 990 B/V6) set at 450 nm (wavelength correction at 540 nm) was used for making the calibration and analyzing the samples.

### 2.4.5 Protein Measurement

For protein concentration determination in the aqueous humor we used the Protein Quantification Kit (Fluka; Cat. No. 77371; Lot. No. 1303129). Five microliters of aqueous humor were diluted to 100 μl with water. Twenty microliters of samples and of standards were load into a 96-well plate and recorded in a plate layout. Samples were treated following the assay procedure described in the kit. A microplate reader (GDV, Italy; model DV 990 B/V6) set at 670 nm was used for making the calibration and analyzing the samples.

### 2.4.6 Leukocytes (PMN) Measurement

For the determination of the number of leukocytes we used a haemocytometer (Improved Neubauer Chamber; Bright-line, Hausser Scientific) and a Polyvar 2 microscope (Reichert-Jung).

### 2.4.7 Leukotriene B₄ (LTB₄) Measurement

For the quantitative determination of LTB₄ concentration in the aqueous humor we used the LTB₄ Immunoassay kit (R&D Systems; Cat. No. KGE006; Lot. No. 243623). 11 μl of aqueous humor were diluted to 110 μl with the calibrator diluent solution provided with the kit. 100 μl of samples and of standards were load into a 96-well plate and recorded in a plate layout. Samples were treated following the assay procedure described in the kit. A microplate reader (GDV, Italy; model DV 990 B/V6) set at 450 nm (wavelength correction at 540 nm) was used for making the calibration and analyzing the samples.

### 2.4.8 Myeloperoxidase (MPO) Measurement

The activity of MPO was measured as previously described by Williams et al.[5] The iris-ciliary bodies were carefully dried, weighed and immersed in 1 ml of hexa-decyl-trimethyl-ammonium bromide solution. Then, the samples were sonicated for 10 sec on ice by a ultrasound homogenizer (HD 2070, Bandelin electronic), freeze-thawed three times, sonicated for 10 sec and centrifuged at 14,000 g for 10 min to remove cellular debris. An aliquot of the supernatant (40-2000 was diluted to 3 ml with the o-dianisidine 2HCl/H₂O₂ solution. The change in absorbance at 460 nm was continuously monitored for 5 min by a spectrophotometer (UV/Vis Spectrometer Lambda EZ 201; Perkin Elmer). The slope of the line (Δ/min) was determined for each sample and used to calculate the number of units of MPO in the tissue as follows:

$$MPOunit \text{/} g = \frac{( Δ \text{/} min ) \cdot {10}^{6}}{ɛ \cdot μ l \cdot mg}$$

were ε=11.3 mM⁻¹.
Values were expressed as units of MPO/g of tissue.

### 2.5 Data Analysis

Pupillary diameter, PGE₂, protein, PMN, and MPO were expressed as mean±SEM. Statistical analysis was performed using one way ANOVA followed by a Newman-Keuls post hoc test. Clinical score was expressed as % of eyes and the statistical analysis was performed using Kruskal-Wallis followed by a Dunn post hoc test. P<0.05 was considered statistically significant in both cases. Prism 4 software (GraphPad Software, Inc.) was used for the analysis and graphs.

### 3. Results

### 3.1 Pupillary Diameter Measurement

The raw data are displayed in Tables T-2 and T-3. No statistical significance was found between the CRT and all the treatments.

### 3.2 Clinical Evaluation

The raw data are displayed in Tables T-4 and T-5. Only the 0.5% LE group showed a significant difference versus CTR (p<0.05).

### 3.3 Prostaglandin E₂ (PGE₂) Measurement

The raw data are displayed in Tables T-6 and T-7. The treatments 0.03% F, 0.5% LE, 0.1% BOL, and 0.5% BOL were statistically significant versus CTR (p<0.05).

### 3.4 Protein Measurement

The raw data are displayed in Tables T-8 and T-9. It has been found a statistical significance for the treatments 0.03% F and 1% BOL vs CTR with p<0.001, and 0.5% BOL vs CTR with p<0.05.

### 3.5 Leukocytes (PMN) Measurement

The raw data are displayed in Tables T-10 and T-11. All the treatments were statistically significant vs CTR (p<0.001).

### 3.6 Leukotriene B₄ (LTB₄) Measurement

All samples were under the limit of quantification (about 0.2 ng/ml) of the assay.

### 3.7 Myeloperoxidase (MPO) Measurement

The raw data are displayed in Tables T-12 and T-13. It has been found a statistical significance for the all the treatments vs CTR with p<0.01 for 0.03% F, and p<0.001 for 0.1% Dex, 0.5% LE, 0.1% BOL, 0.5% BOL and 1% BOL.

### 4. Discussion

The preliminary conclusions from the data generated are:
BOL-303242-X is active in this model.
There was not a large difference between these concentrations of BOL-303242-X and NSAID and steroid positive controls.

There was not a profound dose-response for BOL-303242-X, perhaps because we are at either maximal efficacy or maximal drug exposure at these doses. However, the results show that BOL-303242-X is as effective an anti-inflammatory drug as some of the commonly accepted prior-art steroids or NSAID. Some other very preliminary data (not shown) suggest that BOL-303242-X does not have some of the side effects of corticosteroids.

### 5. REFERENCES

1. Eakins K E (1977). Prostaglandin and non prostaglandin-mediated breakdown of the blood-aqueous barrier. Exp. Eye Res., Vol. 25, 483-498.
2. Neufeld A H, Sears M L (1973). The site of action of prostaglandin E₂ on the disruption of the blood-aqueous barrier in the rabbit eye. Exp. Eye Res., Vol. 17, 445-448.
3. Unger W G, Cole D P, Hammond B (1975). Disruption of the blood-aqueous barrier following paracentesis in the rabbit. Exp. Eye Res., Vol. 20, 255-270.
4. Stjernschantz J (1984). Autacoids and Neuropeptides. In: Sears, M L (ed.) Pharmacology of the Eye. Springer-Verlag, New York, pp. 311-365.
5. Williams R N, Paterson C A, Eakins K E, Bhattacherjee P (1983) Quantification of ocular inflammation: evaluation of polymorphonuclear leukocyte infiltration by measuring myeloperoxidase activity. Curr. Eye Res., Vol. 2, 465-469.

TABLE T-1
Rabbit body weight measured just before the experiment

| Rabbit ID | Sex | Body weight (g) |
|---|---|---|
| A1 | M | 2090 |
| A2 | M | 2140 |
| A3 | M | 2100 |
| A4 | M | 2320 |
| B1 | M | 2270 |
| B2 | M | 2190 |
| B3 | M | 2340 |
| B4 | M | 2300 |
| C1 | M | 2160 |
| C2 | M | 2160 |
| C3 | M | 2280 |
| C4 | M | 2400 |
| D1 | M | 2220 |
| D2 | M | 2200 |
| D3 | M | 2180 |
| D4 | M | 2260 |
| E1 | M | 2170 |
| E2 | M | 2330 |
| E3 | M | 2350 |
| E4 | M | 2300 |
| F1 | M | 2190 |
| F2 | M | 2240 |
| F3 | M | 2120 |
| F4 | M | 2200 |
| G1 | M | 2410 |
| G2 | M | 2270 |
| G3 | M | 2310 |
| G4 | M | 2130 |

| | |
|---|---|
| Mean ± S.D. | 2236.8 ± 89.2 |

TABLE T-2
Raw data of pupillary diameter at −180 min (basal), −5 min (5 min before
the first paracentesis) and at +115 min (5 min before the second
paracentesis), and calculated difference between the value at +115 min
and the value at −180 min.

| | | | Diameter (mm) | | | |
|---|---|---|---|---|---|---|
| Treatment | Rabbit ID | Eye | T1: −180 min | T2: −5 min | T3: +115 min | Δ(T3 − T1) |
| CTR | A1 | DX | 6.0 | 5.5 | 4.0 | −2.0 |
| | | SX | 5.5 | 5.5 | 4.0 | −1.5 |
| | A2 | DX | 6.0 | 6.5 | 4.5 | −1.5 |
| | | SX | 6.0 | 6.5 | 5.0 | −1.0 |
| | A3 | DX | 6.5 | 6.5 | 5.0 | −1.5 |
| | | SX | 6.5 | 6.5 | 5.0 | −1.5 |
| | A4 | DX | 6.0 | 6.5 | 5.0 | −1.0 |
| | | SX | 6.0 | 6.5 | 5.0 | −1.0 |
| 0.03% F | B1 | DX | 5.0 | 6.0 | 4.0 | −1.0 |
| | | SX | 5.0 | 6.0 | 3.5 | −1.5 |
| | B2 | DX | 7.0 | 6.5 | 5.5 | −1.5 |
| | | SX | 6.0 | 7.0 | 5.0 | −1.0 |
| | B3 | DX | 6.0 | 6.5 | 4.5 | −1.5 |
| | | SX | 6.0 | 6.5 | 6.0 | 0.0 |
| | B4 | DX | 5.5 | 6.0 | 5.5 | 0.0 |
| | | SX | 6.0 | 5.5 | 5.0 | −1.0 |
| 0.1% Dex | C1 | DX | 6.0 | 5.5 | 5.5 | −0.5 |
| | | SX | 7.0 | 6.5 | 5.5 | −1.5 |
| | C2 | DX | 5.5 | 6.5 | 6.0 | 0.5 |
| | | SX | 5.5 | 6.0 | 5.5 | 0.0 |
| | C3 | DX | 6.5 | 6.0 | 4.5 | −2.0 |
| | | SX | 6.5 | 6.5 | 5.0 | −1.5 |
| | C4 | DX | 6.5 | 7.0 | 6.0 | −0.5 |
| | | SX | 7.0 | 7.5 | 6.5 | −0.5 |
| 0.5% LE | D1 | DX | 6.0 | 6.0 | 4.5 | −1.5 |
| | | SX | 6.0 | 6.0 | 5.0 | −1.0 |
| | D2 | DX | 6.5 | 6.5 | 5.5 | −1.0 |
| | | SX | 6.5 | 6.5 | 5.5 | −1.0 |
| | D3 | DX | 6.0 | 6.0 | 6.0 | 0.0 |
| | | SX | 6.5 | 6.5 | 6.0 | −0.5 |
| | D4 | DX | 6.5 | 6.5 | 6.0 | −0.5 |
| | | SX | 6.5 | 6.5 | 5.0 | −1.5 |
| 0.1% BOL | E1 | DX | 6.5 | 6.5 | 5.0 | −1.5 |
| | | SX | 6.5 | 6.5 | 6.0 | −0.5 |
| | E2 | DX | 6.5 | 7.0 | 5.0 | −1.5 |
| | | SX | 6.5 | 7.0 | 6.0 | −0.5 |
| | E3 | DX | 7.0 | 7.0 | 6.0 | −1.0 |
| | | SX | 7.5 | 7.5 | 6.5 | −1.0 |
| | E4 | DX | 7.0 | 6.5 | 5.5 | −1.5 |
| | | SX | 7.0 | 7.0 | 5.5 | −1.5 |
| 0.5% BOL | F1 | DX | 8.0 | 8.0 | 6.5 | −1.5 |
| | | SX | 8.0 | 8.0 | 6.5 | −1.5 |
| | F2 | DX | 7.0 | 7.0 | 6.5 | −0.5 |
| | | SX | 7.0 | 7.0 | 6.0 | −1.0 |
| | F3 | DX | 7.5 | 7.5 | 7.0 | −0.5 |
| | | SX | 8.0 | 8.0 | 7.0 | −1.0 |
| | F4 | DX | 7.0 | 7.0 | 6.0 | −1.0 |
| | | SX | 7.5 | 7.0 | 6.5 | −1.0 |
| 1% BOL | G1 | DX | 6.0 | 6.0 | 5.5 | −0.5 |
| | | SX | 6.5 | 6.5 | 5.0 | −1.5 |
| | G2 | DX | 6.0 | 6.5 | 5.0 | −1.0 |
| | | SX | 6.0 | 6.5 | 5.0 | −1.0 |
| | G3 | DX | 6.5 | 7.0 | 5.5 | −1.0 |
| | | SX | 6.5 | 7.0 | 5.0 | −1.5 |
| | G4 | DX | 6.5 | 6.5 | 6.0 | −0.5 |
| | | SX | 6.5 | 6.0 | 6.0 | −0.5 |

TABLE T-3
Difference between the value of pupillary diameter at
T3 = +115 min (5 min before the second paracentesis) and
the value at T1 = −180 min (basal)
(Mean ± SEM).

| | Rabbit | Mean (mm) | | |
|---|---|---|---|---|
| Treatment | Group ID | Δ (T3 − T1) | SEM | n |
| CTR | A | −1.4 | 0.12 | 8 |
| 0.03% F | B | −0.9 | 0.22 | 8 |
| 0.1% Dex | C | −0.8 | 0.30 | 8 |
| 0.5% LE | D | −0.9 | 0.18 | 8 |
| 0.1% BOL | E | −1.1 | 0.16 | 8 |
| 0.5% BOL | F | −1.0 | 0.13 | 8 |
| 1% BOL | G | −0.9 | 0.15 | 8 |

TABLE T-4
Raw data of clinical score at −180 min (basal), −5 min (5 min before
the first paracentesis) and at +115 min (5 min before the second
paracentesis).

| | | | Clinical Score | | |
|---|---|---|---|---|---|
| Treatment | Rabbit ID | Eye | −180 min | −5 min | +115 min |
| CTR | A1 | DX | 0 | 1 | 3 |
| | | SX | 0 | 1 | 3 |
| | A2 | DX | 0 | 0 | 2 |
| | | SX | 0 | 0 | 2 |
| | A3 | DX | 0 | 0 | 3 |
| | | SX | 0 | 0 | 3 |
| | A4 | DX | 0 | 0 | 3 |
| | | SX | 0 | 0 | 3 |
| 0.03% F | B1 | DX | 0 | 0 | 2 |
| | | SX | 0 | 0 | 2 |
| | B2 | DX | 0 | 0 | 2 |
| | | SX | 0 | 0 | 2 |
| | B3 | DX | 0 | 0 | 2 |
| | | SX | 0 | 0 | 2 |
| | B4 | DX | 0 | 0 | 2 |
| | | SX | 0 | 0 | 2 |
| 0.1% Dex | C1 | DX | 0 | 0 | 1 |
| | | SX | 0 | 0 | 1 |
| | C2 | DX | 0 | 0 | 1 |
| | | SX | 0 | 0 | 1 |
| | C3 | DX | 0 | 1 | 3 |
| | | SX | 0 | 1 | 3 |
| | C4 | DX | 0 | 0 | 1 |
| | | SX | 0 | 0 | 1 |
| 0.5% LE | D1 | DX | 0 | 0 | 2 |
| | | SX | 0 | 0 | 2 |
| | D2 | DX | 0 | 0 | 1 |
| | | SX | 0 | 0 | 1 |
| | D3 | DX | 0 | 0 | 1 |
| | | SX | 0 | 0 | 1 |
| | D4 | DX | 0 | 0 | 1 |
| | | SX | 0 | 0 | 1 |
| 0.1% BOL | E1 | DX | 0 | 0 | 2 |
| | | SX | 0 | 0 | 2 |
| | E2 | DX | 0 | 0 | 2 |
| | | SX | 0 | 0 | 2 |
| | E3 | DX | 0 | 0 | 2 |
| | | SX | 0 | 0 | 2 |
| | E4 | DX | 0 | 0 | 3 |
| | | SX | 0 | 0 | 3 |
| 0.5% BOL | F1 | DX | 0 | 0 | 2 |
| | | SX | 0 | 0 | 2 |
| | F2 | DX | 0 | 0 | 1 |
| | | SX | 0 | 0 | 2 |
| | F3 | DX | 0 | 0 | 1 |
| | | SX | 0 | 0 | 1 |
| | F4 | DX | 0 | 0 | 2 |
| | | SX | 0 | 0 | 2 |
| 1% BOL | G1 | DX | 0 | 0 | 2 |
| | | SX | 0 | 0 | 2 |
| | G2 | DX | 0 | 0 | 2 |
| | | SX | 0 | 0 | 2 |
| | G3 | DX | 0 | 0 | 2 |
| | | SX | 0 | 0 | 2 |
| | G4 | DX | 0 | 0 | 2 |
| | | SX | 0 | 0 | 2 |

TABLE T-5
Clinical score expressed as percentage of eyes at −180 min (basal),
−5 min (5 min before the first paracentesis) and at +115 min (5 min
before the second paracentesis).

| | Rabbit | N | Score (%) | | | | |
|---|---|---|---|---|---|---|---|
| Treatment | Group ID | (eyes) | 0 | 1 | 2 | 3 | 4 |
| | | | −180 min | | | | |
| CTR | A | 8 | 100 | — | — | — | — |
| 0.03% F | B | 8 | 100 | — | — | — | — |
| 0.1% Dex | C | 8 | 100 | — | — | — | — |
| 0.5% LE | D | 8 | 100 | — | — | — | — |
| 0.1% BOL | E | 8 | 100 | — | — | — | — |
| 0.5% BOL | F | 8 | 100 | — | — | — | — |
| 1% BOL | G | 8 | 100 | — | — | — | — |
| | | | −5 min | | | | |
| CTR | A | 8 | 75 | 25 | — | — | — |
| 0.03% F | B | 8 | 100 | — | — | — | — |
| 0.1% Dex | C | 8 | 75 | 25 | — | — | — |
| 0.5% LE | D | 8 | 100 | — | — | — | — |
| 0.1% BOL | E | 8 | 100 | — | — | — | — |
| 0.5% BOL | F | 8 | 100 | — | — | — | — |
| 1% BOL | G | 8 | 100 | — | — | — | — |
| | | | +115 min | | | | |
| CTR | A | 8 | — | — | 25 | 75 | — |
| 0.03% F | B | 8 | — | — | 100 | — | — |
| 0.1% Dex | C | 8 | — | 75 | — | 25 | — |
| 0.5% LE | D | 8 | — | 75 | 25 | — | — |
| 0.1% BOL | E | 8 | — | — | 75 | 25 | — |
| 0.5% BOL | F | 8 | — | 37.5 | 62.5 | — | — |
| 1% BOL | G | 8 | — | — | 100 | — | — |

TABLE T-6
Raw data of PGE₂ levels in aqueous humor samples collected at the
second paracentesis

| | | PGE₂ |
|---|---|---|
| Treatment | Sample | (ng/ml) |
| CTR | 2-A1-DX | 3.81 |
| | 2-A1-SX | 2.91 |
| | 2-A2-DX | 4.77 |
| | 2-A2-SX | ¹N/A |
| | 2-A3-DX | 1.46 |
| | 2-A3-SX | 3.00 |
| | 2-A4-DX | 1.87 |
| | 2-A4-SX | 1.88 |
| 0.03% F | 2-B1-DX | 1.04 |
| | 2-B1-SX | 0.75 |
| | 2-B2-DX | 0.85 |
| | 2-B2-SX | 1.11 |
| | 2-B3-DX | 2.11 |
| | 2-B3-SX | 0.93 |
| | 2-B4-DX | 0.61 |
| | 2-B4-SX | 2.11 |
| 0.1% Dex | 2-C1-DX | 2.51 |
| | 2-C1-SX | N/A |
| | 2-C2-DX | 2.32 |
| | 2-C2-SX | N/A |
| | 2-C3-DX | 2.10 |
| | 2-C3-SX | 3.03 |
| | 2-C4-DX | 2.32 |
| | 2-C4-SX | 1.30 |
| 0.5% LE | 2-D1-DX | ²N/D |
| | 2-D1-SX | N/D |
| | 2-D2-DX | N/D |
| | 2-D2-SX | 0.23 |
| | 2-D3-DX | N/D |
| | 2-D3-SX | 0.68 |
| | 2-D4-DX | N/D |
| | 2-D4-SX | 1.10 |
| 0.1% BOL | 2-E1-DX | 1.62 |
| | 2-E1-SX | 1.88 |
| | 2-E2-DX | 2.15 |
| | 2-E2-SX | 0.70 |
| | 2-E3-DX | 1.34 |
| | 2-E3-SX | 1.03 |
| | 2-E4-DX | N/D |
| | 2-E4-SX | N/D |
| 0.5% BOL | 2-F1-DX | 2.31 |
| | 2-F1-SX | 2.59 |
| | 2-F2-DX | N/D |
| | 2-F2-SX | 0.53 |
| | 2-F3-DX | 0.75 |
| | 2-F3-SX | 0.80 |
| | 2-F4-DX | 1.62 |
| | 2-F4-SX | 1.09 |
| 1% BOL | 2-G1-DX | 0.50 |
| | 2-G1-SX | 1.87 |
| | 2-G2-DX | 1.71 |
| | 2-G2-SX | 4.04 |
| | 2-G3-DX | 1.11 |
| | 2-G3-SX | 3.78 |
| | 2-G4-DX | N/D |
| | 2-G4-SX | N/D |

¹N/A = not available
²N/D = not detectable, under the limit of quantification

TABLE T-7
Levels of PGE₂ in aqueous humor samples collected at the second
paracentesis (Mean ± SEM).

| | | Mean | | |
|---|---|---|---|---|
| Treatment | Sample Group | (ng/ml) | SEM | n |
| CTR | A | 2.815 | 0.449 | 7 |
| 0.03% F | B | 1.189 | 0.209 | 8 |
| 0.1% Dex | C | 2.263 | 0.232 | 6 |
| 0.5% LE | D | 0.672 | 0.250 | 3 |
| 0.1% BOL | E | 1.452 | 0.221 | 6 |
| 0.5% BOL | F | 1.384 | 0.306 | 7 |
| 1% BOL | G | 2.168 | 0.586 | 6 |

TABLE T-8
Raw data of protein levels in aqueous humor samples collected at the
second paracentesis

| | | Protein |
|---|---|---|
| Treatment | Sample | (mg/ml) |
| CTR | 2-A1-DX | 50.24 |
| | 2-A1-SX | 53.51 |
| | 2-A2-DX | 28.73 |
| | 2-A2-SX | ¹N/A |
| | 2-A3-DX | 40.09 |
| | 2-A3-SX | 30.84 |
| | 2-A4-DX | 41.79 |
| | 2-A4-SX | 30.35 |
| 0.03% F | 2-B1-DX | 20.78 |
| | 2-B1-SX | 28.80 |
| | 2-B2-DX | N/A |
| | 2-B2-SX | 23.41 |
| | 2-B3-DX | 20.21 |
| | 2-B3-SX | 17.53 |
| | 2-B4-DX | 15.12 |
| | 2-B4-SX | 20.52 |
| 0.1% Dex | 2-C1-DX | 31.31 |
| | 2-C1-SX | N/A |
| | 2-C2-DX | 31.81 |
| | 2-C2-SX | N/A |
| | 2-C3-DX | 35.95 |
| | 2-C3-SX | 37.15 |
| | 2-C4-DX | 32.12 |
| | 2-C4-SX | 32.40 |
| 0.5% LE | 2-D1-DX | 36.14 |
| | 2-D1-SX | 39.10 |
| | 2-D2-DX | 34.69 |
| | 2-D2-SX | 26.10 |
| | 2-D3-DX | 26.30 |
| | 2-D3-SX | 28.16 |
| | 2-D4-DX | 40.90 |
| | 2-D4-SX | 39.85 |
| 0.1% BOL | 2-E1-DX | 34.87 |
| | 2-E1-SX | 34.41 |
| | 2-E2-DX | 31.14 |
| | 2-E2-SX | 22.82 |
| | 2-E3-DX | 29.46 |
| | 2-E3-SX | 31.69 |
| | 2-E4-DX | 35.70 |
| | 2-E4-SX | 49.25 |
| 0.5% BOL | 2-F1-DX | 33.98 |
| | 2-F1-SX | 33.65 |
| | 2-F2-DX | 19.99 |
| | 2-F2-SX | 27.11 |
| | 2-F3-DX | 19.72 |
| | 2-F3-SX | 36.35 |
| | 2-F4-DX | 27.71 |
| | 2-F4-SX | 32.24 |
| 1% BOL | 2-G1-DX | 20.99 |
| | 2-G1-SX | 21.48 |
| | 2-G2-DX | 15.11 |
| | 2-G2-SX | 20.28 |
| | 2-G3-DX | 20.94 |
| | 2-G3-SX | 21.89 |
| | 2-G4-DX | 20.03 |
| | 2-G4-SX | 30.76 |

¹N/A = not available

TABLE T-9
Protein levels in aqueous humor samples collected at the second
paracentesis (Mean ± SEM).

| | | Mean | | |
|---|---|---|---|---|
| Treatment | Sample Group | (mg/ml) | SEM | n |
| CTR | A | 39.364 | 3.754 | 7 |
| 0.03% F | B | 20.910 | 1.648 | 7 |
| 0.1% Dex | C | 33.457 | 1.001 | 6 |
| 0.5% LE | D | 33.905 | 2.190 | 8 |
| 0.1% BOL | E | 33.667 | 2.655 | 8 |
| 0.5% BOL | F | 28.844 | 2.249 | 8 |
| 1% BOL | G | 21.435 | 1.529 | 8 |

TABLE T-10
Raw data of PMN numbers in aqueous humor samples collected at the
second paracentesis

| | | PMN |
|---|---|---|
| Treatment | Sample | (number/μl) |
| CTR | 2-A1-DX | 90 |
| | 2-A1-SX | 80 |
| | 2-A2-DX | 70 |
| | 2-A2-SX | ¹N/A |
| | 2-A3-DX | 70 |
| | 2-A3-SX | 80 |
| | 2-A4-DX | 50 |
| | 2-A4-SX | 40 |
| 0.03% F | 2-B1-DX | 50 |
| | 2-B1-SX | 40 |
| | 2-B2-DX | N/A |
| | 2-B2-SX | 20 |
| | 2-B3-DX | 10 |
| | 2-B3-SX | 40 |
| | 2-B4-DX | 30 |
| | 2-B4-SX | 20 |
| 0.1% Dex | 2-C1-DX | 20 |
| | 2-C1-SX | N/A |
| | 2-C2-DX | 20 |
| | 2-C2-SX | N/A |
| | 2-C3-DX | 50 |
| | 2-C3-SX | 40 |
| | 2-C4-DX | 20 |
| | 2-C4-SX | 30 |
| 0.5% LE | 2-D1-DX | N/A |
| | 2-D1-SX | N/A |
| | 2-D2-DX | 40 |
| | 2-D2-SX | 20 |
| | 2-D3-DX | 20 |
| | 2-D3-SX | 30 |
| | 2-D4-DX | 40 |
| | 2-D4-SX | 20 |
| 0.1% BOL | 2-E1-DX | N/A |
| | 2-E1-SX | 20 |
| | 2-E2-DX | 40 |
| | 2-E2-SX | 50 |
| | 2-E3-DX | 20 |
| | 2-E3-SX | 20 |
| | 2-E4-DX | 20 |
| | 2-E4-SX | N/A |
| 0.5% BOL | 2-F1-DX | 40 |
| | 2-F1-SX | 20 |
| | 2-F2-DX | 20 |
| | 2-F2-SX | 10 |
| | 2-F3-DX | 10 |
| | 2-F3-SX | 10 |
| | 2-F4-DX | 20 |
| | 2-F4-SX | 40 |
| 1% BOL | 2-G1-DX | 30 |
| | 2-G1-SX | 20 |
| | 2-G2-DX | 30 |
| | 2-G2-SX | 40 |
| | 2-G3-DX | 20 |
| | 2-G3-SX | 30 |
| | 2-G4-DX | 40 |
| | 2-G4-SX | 20 |

¹N/A = not available

TABLE T-11
PMN numbers in aqueous humor samples collected at the second
paracentesis (Mean ± SEM).

| | | Mean | | |
|---|---|---|---|---|
| Treatment | Sample Group | (number/μl) | SEM | n |
| CTR | A | 68.571 | 6.701 | 7 |
| 0.03% F | B | 30.000 | 5.345 | 7 |
| 0.1% Dex | C | 30.000 | 5.164 | 6 |
| 0.5% LE | D | 28.333 | 4.014 | 6 |
| 0.1% BOL | E | 28.333 | 5.426 | 6 |
| 0.5% BOL | F | 21.250 | 4.407 | 8 |
| 1% BOL | G | 28.750 | 2.950 | 8 |

TABLE T-12
Raw data of MPO activity in iris-ciliary body samples collected after
the second paracentesis.

| | | Iris-ciliary body | ¹Volume | | MPO |
|---|---|---|---|---|---|
| Treatment | Sample | weight (mg) | (μl) | ²Δ/min | Unit/g |
| CTR | A1-DX | 41.7 | 40 | 0.021 | 1.11 |
| | A1-SX | 42.3 | 40 | 0.024 | 1.26 |
| | A2-DX | 46.6 | 40 | 0.039 | 1.85 |
| | A2-SX | 40.5 | 40 | 0.037 | 2.02 |
| | A3-DX | 48.9 | 40 | 0.075 | 3.39 |
| | A3-SX | 51.1 | 40 | 0.049 | 2.12 |
| | A4-DX | 36.6 | 40 | 0.013 | 0.79 |
| | A4-SX | 38.8 | 40 | 0.019 | 1.08 |
| 0.03% F | B1-DX | 39.5 | 100 | 0.049 | 1.10 |
| | B1-SX | 42.7 | 100 | 0.082 | 1.70 |
| | B2-DX | 34.1 | 100 | 0.013 | 0.34 |
| | B2-SX | 36.6 | 100 | 0.031 | 0.75 |
| | B3-DX | 45.6 | 100 | 0.038 | 0.74 |
| | B3-SX | 38.0 | 100 | 0.027 | 0.63 |
| | B4-DX | 40.1 | 100 | 0.033 | 0.73 |
| | B4-SX | 42.6 | 100 | 0.061 | 1.27 |
| 0.1% Dex | C1-DX | 36.4 | 100 | 0.029 | 0.71 |
| | C1-SX | 45.8 | 100 | 0.031 | 0.60 |
| | C2-DX | 42.9 | 100 | 0.064 | 1.32 |
| | C2-SX | 42.7 | 100 | 0.023 | 0.48 |
| | C3-DX | 43.0 | 100 | 0.019 | 0.39 |
| | C3-SX | 46.8 | 100 | 0.024 | 0.45 |
| | C4-DX | 42.3 | 100 | 0.023 | 0.48 |
| | C4-SX | 36.1 | 100 | 0.021 | 0.51 |
| 0.5% LE | D1-DX | 38.9 | 200 | 0.026 | 0.30 |
| | D1-SX | 44.7 | 200 | 0.053 | 0.51 |
| | D2-DX | 35.9 | 200 | 0.067 | 0.81 |
| | D2-SX | 40.7 | 200 | 0.055 | 0.60 |
| | D3-DX | 46.3 | 200 | 0.076 | 0.73 |
| | D3-SX | 41.9 | 200 | 0.096 | 1.01 |
| | D4-DX | 46.7 | ³N/A | N/A | N/A |
| | D4-SX | 32.9 | N/A | N/A | N/A |
| 0.1% BOL | E1-DX | 43.6 | 100 | 0.051 | 1.04 |
| | E1-SX | 37.2 | 100 | 0.042 | 1.00 |
| | E2-DX | 32.6 | 100 | 0.042 | 1.14 |
| | E2-SX | 37.4 | 100 | 0.045 | 1.06 |
| | E3-DX | 36.2 | 100 | 0.050 | 1.22 |
| | E3-SX | 45.1 | 100 | 0.031 | 0.61 |
| | E4-DX | 30.4 | 100 | 0.036 | 1.05 |
| | E4-SX | 42.3 | 100 | 0.031 | 0.65 |
| 0.5% BOL | F1-DX | 45.8 | 100 | 0.044 | 0.85 |
| | F1-SX | 38.2 | 100 | 0.040 | 0.93 |
| | F2-DX | 34.9 | 100 | 0.031 | 0.79 |
| | F2-SX | 42.0 | 100 | 0.049 | 1.03 |
| | F3-DX | 39.1 | 100 | 0.033 | 0.75 |
| | F3-SX | 40.6 | 100 | 0.034 | 0.74 |
| | F4-DX | 36.2 | 100 | 0.022 | 0.54 |
| | F4-SX | 39.5 | 100 | 0.026 | 0.58 |
| 1% BOL | G1-DX | 32.4 | 100 | 0.024 | 0.66 |
| | G1-SX | 43.1 | 100 | 0.033 | 0.68 |
| | G2-DX | 30.6 | 100 | 0.017 | 0.49 |
| | G2-SX | 39.9 | 100 | 0.018 | 0.40 |
| | G3-DX | 41.3 | 100 | 0.016 | 0.34 |
| | G3-SX | 44.9 | 100 | 0.052 | 1.02 |
| | G4-DX | 36.6 | 100 | 0.013 | 0.31 |
| | G4-SX | 36.9 | 100 | 0.018 | 0.43 |

¹Volume = aliquot (μl) of the supernatant diluted to 3 ml for the analysis.
²Δ/min = mean of the slope of the line recorded every 15 sec for 5 min
³N/A = not available

TABLE T-13
MPO activity in iris-ciliary body samples collected after the second
paracentesis (Mean ± SEM).

| | | Mean | | |
|---|---|---|---|---|
| Treatment | Sample Group | MPO Unit/g | SEM | n |
| CTR | A | 1.703 | 0.297 | 8 |
| 0.03% F | B | 0.906 | 0.151 | 8 |
| 0.1% Dex | C | 0.618 | 0.106 | 8 |
| 0.5% LE | D | 0.661 | 0.102 | 6 |
| 0.1% BOL | E | 0.971 | 0.079 | 8 |
| 0.5% BOL | F | 0.775 | 0.058 | 8 |
| 1% BOL | G | 0.542 | 0.083 | 8 |

### TESTING 2: Effect of BOL-303242-X on Inhibiting IL-113-Induced Cytokine Expression in Human Corneal Epithelial Cells

### 1. Background/Rationale

Levels of cytokines associated with immune cells are direct indications of activity of these cells in an inflammatory condition. Reduced levels of these cytokines indicate a positive therapeutic effect on inflammation of a test compound. This study was designed to determine the effect of BOL-303242-X on IL-1β-induced cytokine production in human corneal epithelial cells (“HCECs”).

### 1. Purpose

To determine the effects of BOL-303242-X on IL-1β-stimulated cytokine expression in primary human corneal epithelial cells using a 30-cytokine Luminex kit. Dexamethasone was used as a control.

### 3. Experimental Design

Primary HCECs were seeded in 24-well plates. After 24 h, cells were treated with vehicle, IL-1β, IL-1β+dexamethasone, or IL-1β+BOL-303242-X in basic EpiLife medium for 18 h (Table T-14). Each treatment was performed in triplicate. Media were collected and used for determination of cytokine content using a 30-cytokine Luminex kit. Cell viability was determined by alamarBlue assay (LP06013).

| | | Day 2: cells were treated with the test | |
|---|---|---|---|
| Group* | Day 1 | agents in basic EpiLife medium for 18 h | Day 3 |
| 1 | Cells | Control (0.1% DMSO) | Media for |
| 2 | were | 10 ng/ml IL-1β | Luminex |
| 3 | seeded | 10 ng/ml IL-1β + 1 nM dexamethasone | assays; |
| 4 | in | 10 ng/ml IL-1β + 10 nM dexamethasone | cells for |
| 5 | 24-well | 10 ng/ml IL-1β + 100 nM dexamethasone | cell |
| 6 | plates | 10 ng/ml IL-1β + 1 μM dexamethasone | viability |
| 7 | (5 × | 10 ng/ml IL-1β + 10 μM dexamethasone | assay |
| 8 | 10⁵/ | 10 ng/ml IL-1β + 1 nM BOL-303242-X | |
| 9 | well in | 10 ng/ml IL-1β + 10 nM BOL-303242-X | |
| 10 | 0.5 ml | 10 ng/ml IL-1β + 100 nM BOL-303242-X | |
| 11 | medi- | 10 ng/ml IL-1β + 1 μM BOL-303242-X | |
| 12 | um) in | 10 ng/ml IL-1β + 10 μM BOL-303242-X | |
| | EpiLife | | |
| | medium | | |

*triplicate wells per group
Dexamethasone:
Lot Number: 016K14521
Parent MW: 392.46
Parent:Total MW Ratio = 1.0
BOL-303242-X:
Lot Number: 6286
Parent MW: 462.48
Parent:Total MW Ratio = 1.0

### 4. Data Analysis

Median fluorescence intensity (WI) was used to obtain the concentration of each cytokines in pg/ml based on the standard curve of each cytokine assayed by Luminex. The linear range of the standard curve for each cytokine was used for determination of cytokine concentration. Duplicate values for each sample were averaged. Data were expressed as mean±SD. Statistical analysis was performed using one-way ANOVA-Dunnett's test, and P<0.05 was considered statistically significant.

### 5. Results

No statistically significant effect on cellular metabolic activity (as measured by alamarBlue assay) was observed with the various treatments.

Substantial amounts of 16 out of 30 cytokines tested were detected in this study and 13 out of 14 cytokines detected were stimulated by 10 ng/ml IL-1β (Table T-14). IL-1β was excluded from analysis because it was the stimulus. IL-1ra was excluded because the MFI was not within the standard range.

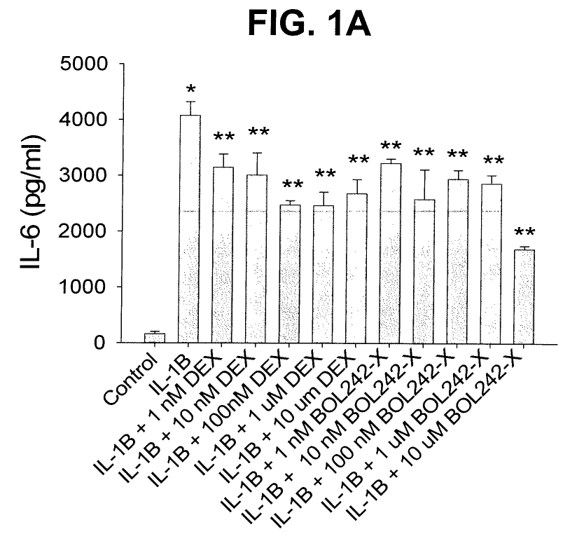

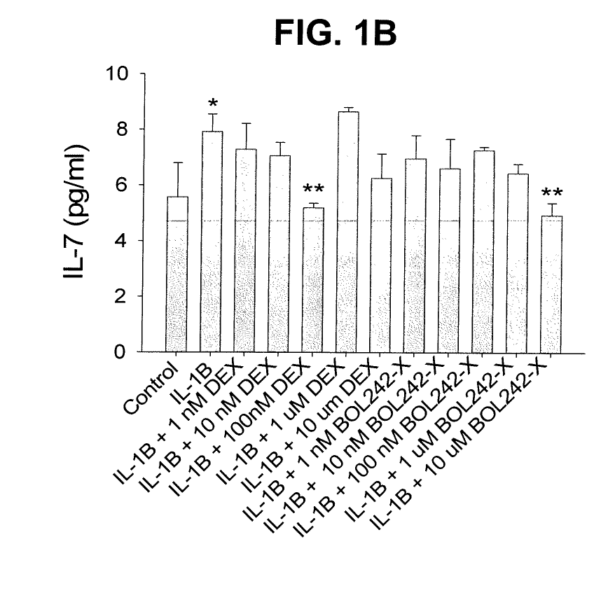

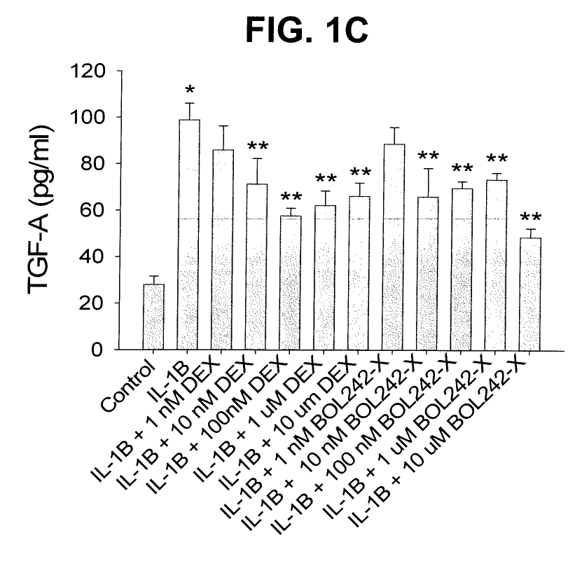

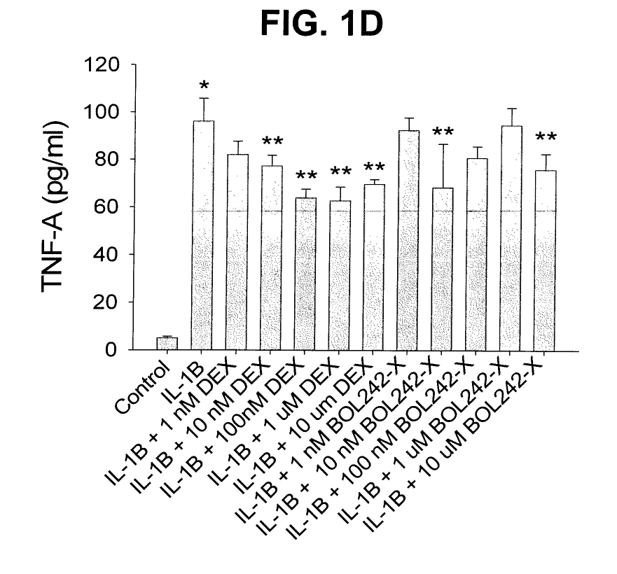

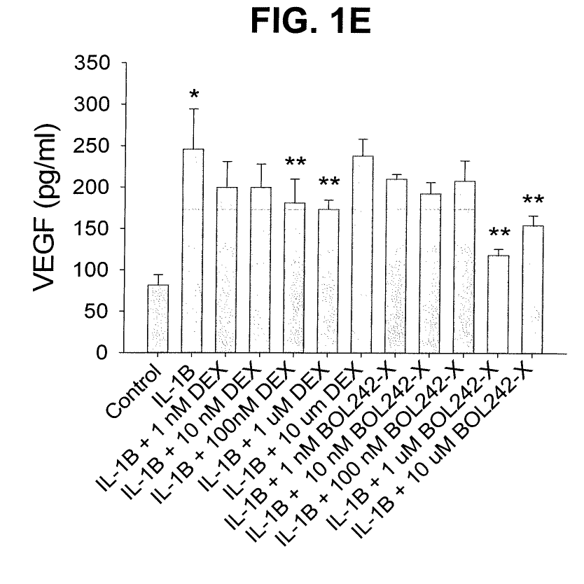

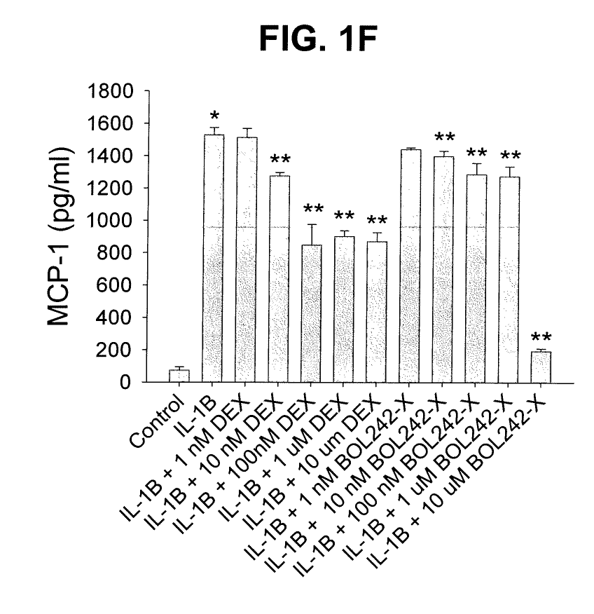

Dexamethasone and BOL-303242-X significantly inhibited IL-1β-stimulated cytokine production with comparable potency on 6 cytokines (IL-6, IL-7, MCP-1, TGF-α, TNF-α and VEGF), and a significant inhibitory effect was observed at 1 nM on IL-6 and at 10 nM on MCP-1, TGF-α and TNF-α (Table T-14 and FIGS. 1A-1F).

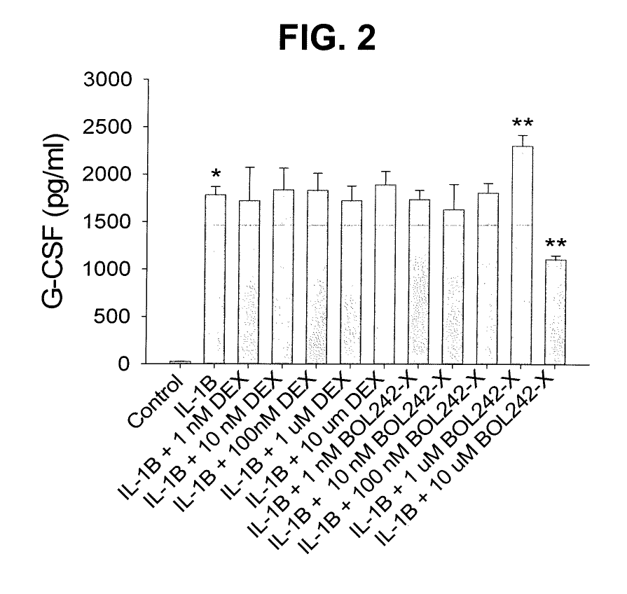

BOL-303242-X also significantly inhibited IL-1β-stimulated G-CSF production with better potency compared to dexamethasone, and a significant inhibitory effect was observed at 10 μg/mlby BOL-303242-X while no significant effect was observed by dexamethasone on this cytokine (FIG. 2).

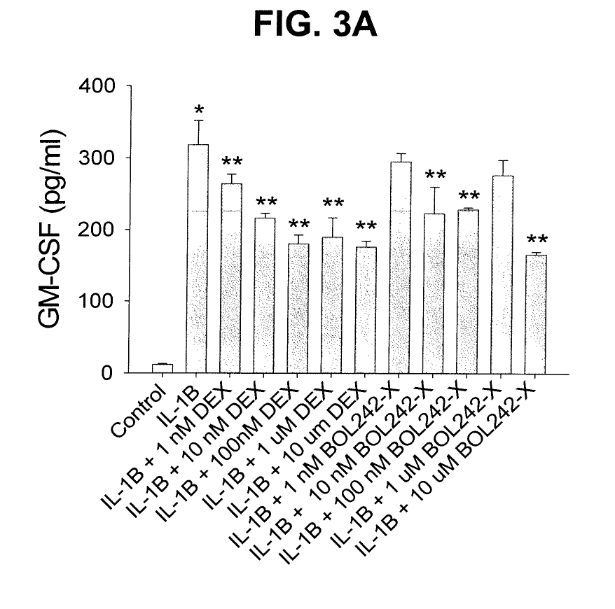

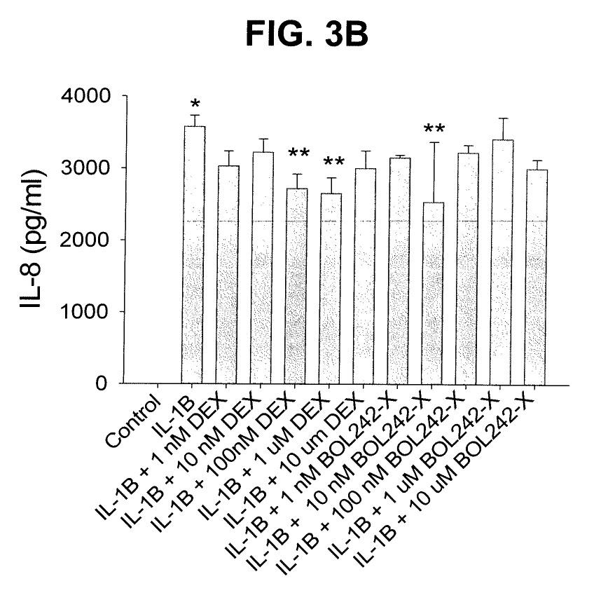

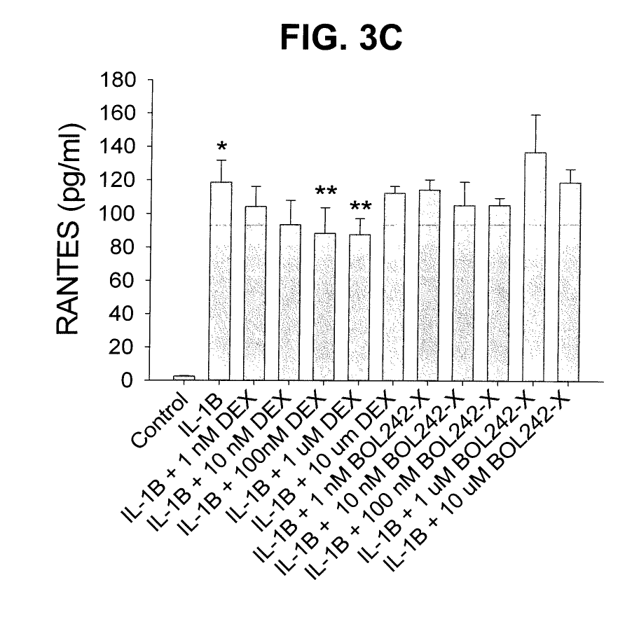

BOL-303242-X also significantly inhibited IL-1β-stimulated cytokine production with less potency compared to dexamethasone on 3 cytokines (GM-CSF, IL-8, and RANTES). A significant inhibitory effect was observed at 1 nM by dexamethasone and at 10 nM by BOL-303242-X on GM-CSF. A significant inhibitory effect was observed at 1 μM by dexamethasone on RANTES while no significant effect was observed by BOL-303242-X on this cytokine (FIGS. 3A-3C).

### 6. CONCLUSION

BOL-303242-X and dexamethasone have comparable potency for inhibition of IL-1β-stimulated cytokine production in HCECs for the cases of IL-6, IL-7, TGF-α, TNF-α, VGEF, and MCP-1. BOL-303242-X is more potent than dexamethasone in inhibiting IL-1β-stimulated production of G-CSF in HCECs. BOL-303242-X is somewhat less potent than dexamethasone in inhibiting IL-1β-stimulated production of GM-CSF, IL-8, and RANTES in HCECs.

TABLE T-14
Inhibition of IL-1β stimulated cytokine production by dexamethasone
and BOL-303242-X in primary human corneal epithelial cells

| | Stimulated | | | | | | | | | | |
|---|---|---|---|---|---|---|---|---|---|---|---|
| | by IL- | Inhibited by | | | | | Inhibited by | | | | |
| Cytokines | 1β | dexamethasone (μM) | | | | | BOL-303242-X (μM) | | | | |
| detected* | (10 ng/ml) | 0.001 | 0.01 | 0.1 | 1 | 10 | 0.001 | 0.01 | 0.1 | 1 | 10 |
| G-CSF | X | | | | | | | | | | X |
| GM-CSF | X | X | X | X | X | X | | X | X | | X |
| IL-1α | X | | | | | | | | | | |
| IL-6 | X | X | X | X | X | X | X | X | X | X | X |
| IL-7 | X | | | X | | | | | | | X |
| IL-8 | X | | | X | X | | | X | | | |
| IP-10 | X | | | | | | | | | | |
| MCP-1 | X | | X | X | X | X | | X | X | X | X |
| MIP-1α | | | | | | | | | | | |
| MIP-1β | X | | | | | | | | | | |
| RANTES | X | | | | X | X | | | | | |
| TGF-α | X | | X | X | X | X | | X | X | X | X |
| TNF-α | X | | X | X | X | X | | X | | | X |
| VEGF | X | | | X | X | | | | | X | X |

Notes:
*EGF, Eotaxin, Fractalkine, IFNγ, IL-10, IL-12p40, IL-12p70, IL-13, IL15, IL-17, IL-2, IL-4, IL-5, sCD40L were not detected. IL-1β was excluded from analysis because it was the stimulus. IL-1ra was excluded because the MFI was out of range of the standards.

Testing 3: Myocilin Expression in and Release from Trabecular Meshwork Cells Upon Treatment with Dexamethasone or BOL-303242-X

### Materials and Methods

### TM Cells and Culture Media

All animal procedures were in accordance with the ARVO (Association for Research in Vision and Ophthalmology) resolution on animal care. Eyes from freshly killed, healthy rhesus monkeys (Macaca mulatta), obtained from Lonza (Walkersville, Md.), were transported in CO₂-independent medium on ice, and processed approximately 40 hours post-enucleation. Following removal of iris, lens, and the bulk of the ciliary body, opercula (an anatomical feature of monkey TM) were stripped from anterior segment quadrants. Using fine scissors, strips of TM were excised, and subdivided TM fragments were explanted to multiwell plates containing growth medium (described below) and incubated with Cytodex-3 gelatin-coated beads (Sigma Chemical Company, St. Louis, Mo.). The beads attach to the explants within hours and provide additional substrate area for out-migration of cells. Proliferating TM cells colonize additional beads and also “spill” onto the tissue culture plastic and form colonies. After several days, the original TM explants and beads were transferred to new wells, generating additional primary cultures. Subconfluent monolayers of cells on tissue culture plastic were passed from 12-well plates to 35- or 60-mm dishes using a Collagenase-Dispase (Roche Applied Bioscience, Indianapolis, Ind.). Second- or third-passage subcultures were finally harvested enzymatically as above, and the cells were counted and cryopreserved in liquid nitrogen.

The medium for initiating and expanding cultures of TM (proliferation medium) was Human Endothelial Serum-Free Medium (“HESFM”; InVitrogen, Carlsbad, Calif.), containing the following supplements: fetal bovine serum (“FBS”; 1% (v/v); Hyclone, Logan, Utah); endothelial cell growth supplement (25 μg/ml; BD Biosciences, San Jose, Calif.); heparin (2.5 μg/ml; Sigma); taurine (3.2 μM; Sigma); fatty acid-albumin complex (200 mg/L; Invitrogen); ascorbic acid phosphate (0.1 mM; Wako Pure Chemicals, Richmond, Va.); human transferrin (25 mg/L; Sigma); human fetuin (0.1 mg/ml; Sigma); glucose (1.5 g/L; Sigma); fructose (0.33 g/L; Sigma); glutathione (5 μg/ml; Sigma); hydrocortisone (14 nM; Sigma); and penicillin-streptomycin (InVitrogen) as antibiotic additive.

For each study, up to nine TM cell strains, each derived from an individual monkey, were tested separately. Cells were thawed and seeded into 12- or 48-well clusters (Falcon, BD Biosciences; 150,000 and 30,000 cells/well, respectively) in proliferation medium. When cells were 75% to 90% confluent proliferation medium was replaced by a 5:4 mixture of HESFM and Dulbecco's MEM, respectively, supplemented with 10% FBS, with added taurine, ascorbic acid phosphate, glutathione, and antibiotic as for proliferation medium (above), and with 2.72 g/L glucose and 1.72 g/L fructose. At confluence, the medium was changed to Dulbecco's MEM, containing 10% FBS⁴⁰, ascorbic acid phosphate, antibiotic, 2.72 g/L glucose and 1.72 g/L fructose. Cells were maintained as stable, confluent monolayers in this latter medium for 4 to 7 days before experimental treatments commenced.

TM Cell Treatments with DEX and BOL-303242-X

TM cell strains from nine different individual monkeys were used to directly compare the responses to DEX and BOL-303242-X. Cells in triplicate sample wells (24-well clusters) were incubated with DEX (Sigma) in individual studies alongside corresponding cell samples exposed to BOL-303242-X; drug concentrations ranged from 3 to 300 nM.

All treatments, including media for vehicle control samples, contained a final DMSO concentration of 0.1% (v/v) across the concentration ranges selected. Treatments lasted 96 hours, with one exchange of medium on the third treatment day. The final 48-hr conditioned media (“CM”) samples were collected in their entirety (0.5 ml), centrifuged briefly to remove particulates, aliquoted, and stored at −20° C. until thawed for analysis.

### Cell Metabolic Activity Assay

A modification of previously described methods⁴⁰ was employed to evaluate cell metabolic activity, an index of cell viability. After collection of CM samples, cells were briefly rinsed in modified Hanks balanced salt solution containing Ca⁺⁺ and Mg⁺⁺ (“MHBSS”), and then 0.0025% (w/v) resazurin (Sigma) in MHBSS was added to sample wells. Plates were incubated (37° C., 5% CO₂, 95% humidity) for 90 minutes, after which fluorescence (Excitation 560 nm, Emission 590 nm) was read (Victor 3V Multilabel Counter, Wallac, Turku, Finland). As a positive control for decreased cellular metabolic reduction of resazurin, in each plate an additional well of vehicle control-treated cells was preincubated with 0.06% hydrogen peroxide (Fisher, Atlanta, Ga.) in MHBSS.

### Western Blot Analysis

Undiluted CM was combined with denaturing 4× sample buffer containing 2% SDS, and samples were loaded at equivalent protein content onto 4-20% Tris-HCl polyacrylamide gels (BioRad, Hercules, Calif.). After electrophoresis, proteins underwent wet transfer to 0.2 mm nitrocellulose (BioRad) for immunoblotting. The filters were blocked with 5% (w/v) nonfat dry milk (BioRad) in Tris-buffered saline plus 0.02% (v/v) Tween-20 (“TBST”; Tween-20 from Calbiochem, San Diego, Calif.), and incubated with a 1:2000 dilution (from 200 μg/ml) of goat anti-recombinant human myocilin antibody (R&D Systems, Minneapolis, Minn.) in blocking buffer, overnight at 4° C. After washing in TBST, the filters were incubated with a 1:25,000 dilution (from 0.8 mg/ml) of horseradish peroxide-conjugated mouse anti-goat IgG (H+L) (Pierce Biotechnology, Rockford, Ill.) in blocking buffer, for 90 minutes at room temperature. After washing in TBST, the blots were developed in SuperSignal® West Dura Extended Duration Substrate (Pierce) for chemiluminescent detection. Bands corresponding to myocilin were digitally captured and stored using a FluorChem Imager (Alpha-Innotech, San Leandro, Calif.), with all blots receiving equal exposure/capture times. The imager system software was then used to calculate pixel density for equivalent rectangular areas incorporating the bands.

Quantitative Real Time Reverse Transcriptase-Polymerase Chain Reaction (qRT-PCR)

Following triplicate treatments with DEX, PA, BOL-303242-X, or vehicle control medium, cultured TM cells prepared in 6-well clusters were lysed, and total RNA was isolated using the RNeasy Plus MiniKit from Qiagen (Valencia, Calif.) according to the manufacturer's instructions. After quantification of purified total RNA (Quant-iT RNA Assay kit, Molecular Probes, Eugene, Oreg.), equivalent amounts of this RNA were apportioned to generate first-strand cDNAs for each treatment sample, using random primers, (Affinity Script, Stratagene, La Jolla, Calif.). Oligonucleotide myocilin primers, designed based on the cynomolgus MYOC gene, and fluorescent Taqman probe (Applied Biosystems, Foster City, Calif.) were used for PCR amplification. Equal amounts of total RNA-equivalent mass (approximate range 250-1000 μg) reactant cDNA were added to the PCR Master Mix (Stratagene) and myocilin primers/Taqman probe. Amplification was performed in a thermocycler (Mx3005P, Stratagene), with an initial denaturation step at 95° C. for 10 min, followed by 40 cycles of 95° C. for 15 sec and 60° C. for 1 min for extension. Every run included standard controls (i.e., either without reverse transcriptase or lacking template). Relative quantities of myocilin mRNA abundance were determined using differences in threshold cycles (“Ct”) between vehicle control and drug treatments. Each sample was analyzed in triplicate wells, and the corresponding values averaged for further quantitative analysis. Myocilin mRNA abundance, expressed in proportion to vehicle control-treated samples, was calculated using the Mx3005P software.

### Data Analysis and Statistical Methods

Data underwent Box-Cox transformations for one- or two-way analysis of variance (ANOVA), followed by the Tukey-Kramer test, using JMP software (SAS, Cary, N.C.). The specific transformations used for analyses are mentioned in the figure legends. For each set of triplicate samples from the individual monkey TM cell strains tested, Western blot densitometry values for myocilin protein detected in CM (as geometric means), and relative abundance of myocilin mRNA (as geometric means), were plotted as a function of drug concentrations. P-values less than 0.05 were considered statistically significant. Dose-response curve data were fitted to a re-parameterized four-parameter logistic equation using similar methodology to that previously described, and these equations permitted estimation of the EC₅₀ values±95% confidence intervals, for each drug treatment.

### Results

### In Vitro Properties of Monkey TM Cells

Rhesus monkey TM cells demonstrated robust proliferation both in primary explants and during early passage. General cellular morphology and the uniform cobblestone pattern of the monolayers, consistent with TM cells propagated from young human donors and cynomolgus monkeys reported in the prior art, were maintained in confluent subcultures used for these studies.

Effects of DEX and BOL-303242-X on Myocilin Protein in Monkey TM Cell CM

Myocilin protein was released to CM by rhesus monkey TM cells, and was detected in Western blots as a single thick band—probably a fused doublet—at the expected molecular size, approximately 55 kDa, as previously noted in Western blots of CM from DEX-treated human TM cells and of monkey aqueous fluid. With exposure to increasing concentrations of BOL-303242-X or DEX, immunoreactive bands of higher density could be discerned by visual inspection alone. It is important to note though, that DEX induced higher expression of myocilin than the BOL-303242-X at high doses, suggesting a partial agonist activity for the BOL-303242-X on myocilin gene expression.

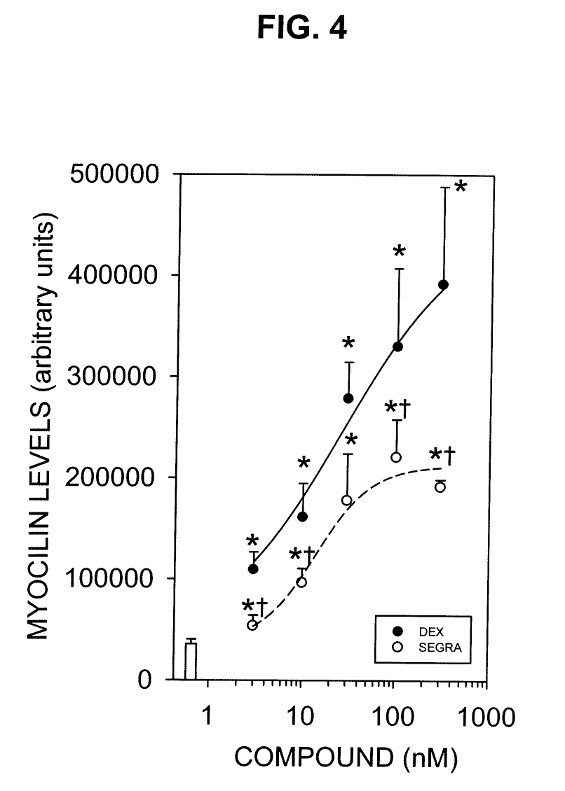

FIG. 3 shows the effects of DEX and BOL-303242-X on the amount of accumulated myocilin protein released into the CM during the second 48-hour treatment period. Whereas both compounds increased myocilin concentrations in a dose-dependent manner, the amounts of myocilin produced, and released into the medium, by BOL-303242-X, at all doses studied, are less for BOL-303242-X than for DEX. As illustrated in FIG. 4 for one monkey TM cell strain, the full range of DEX treatments gave statistically significant effects compared to vehicle control (FIG. 4, solid symbols). (Note that 100 nM, corresponding to the topical dose routinely used for DEX in clinical applications, is also commonly invoked to assess steroid responsiveness in vitro⁵⁵.) Within the dose range utilized, maximal efficacy of DEX was achieved at 300 nM; for one of the monkey TM cell strains that was tested this concentration of DEX yielded a myocilin protein level 1233% (ca. 11-fold) over control. In several strains tested, no clear plateau in the high concentration range was identified for the DEX dose-response curve (FIG. 4). While BOL-303242-X also increased myocilin accumulation in the CM of the monkey TM cells throughout the concentration range tested (FIG. 4, open symbols), the maximal efficacy computed across all nine TM cells strains, was about 50% of that observed after DEX treatment (Table T-15). In fact, the dose response curve for the BOL-303242-X showed clear indication of a plateau approaching the high dose concentration range, indicating that the compound had reached its maximal efficacy. The partial agonism of BOL-303242-X was further demonstrated by the statistically significant differences observed between DEX and BOL-303242-X at 3, 10, 100, and 300 nM (indicated by daggers in FIG. 4). With respect to potency, DEX and BOL-303242-X displayed EC₅₀s of 14.58 and 20.96 nM, respectively (Table T-16). These differences were not statistically significant, with overlapping 95% confidence limits for the estimates (Table T-16). In experiments conducted over three months, the responses of the 9 monkey TM isolates were similar and very reproducible; the inter-isolate variabilities for the EC₅₀s were 18.20% and 20.40% for DEX and BOL-303242-X, respectively (Table T-16). The results indicate that BOL-303242-X, as a partial GC agonist, induced significantly lower levels of myocilin protein to be released by cultured monkey TM cells, compared to the model GC DEX.

TABLE T-15
Partial agonism of BOL-303242-X in comparison with DEX.
Estimated efficacy at 300 nM, for inducing
myocilin protein expression in cultured monkey TM cells.

| | Efficacy ± SE¹ | | Coef. |
|---|---|---|---|
| | (weighted average; | 95% Confidence | of Variation |
| Compound | %) | Limits for Efficacy | (%) |
| DEX | 100 ± 6.09 | 88.07-111.93 | 18.27 |
| BOL-303242-X | 53.12 ± 2.20 | 48.81-57.43 | 12.42 |

¹The efficacies presented are calculated as the weighted averages for each experiment normalized to DEX (100%). The inverse of the variance for each strain is used for the weight.
²Data are averaged from nine experiments (one per strain) that were conducted over a period of three months.

TABLE T-16
Comparison of the potency of DEX and BOL-303242-X on
expression of myocilin protein by cultured monkey TM cells.
Compilation of data from two independent dose-response studies,
using nine monkey TM cell strains.

| | | | Coefficient of |
|---|---|---|---|
| | | 95% Confidence | Variation |
| Compound | EC₅₀ ± SE (nM)* | Limits for EC₅₀ | (%) |
| DEX | 14.58 ± 2.65 | 10.21-20.83 | 18.20 |
| BOL-303242-X | 20.96 ± 4.28 | 14.05-31.26 | 20.40 |

*The EC₅₀s presented were calculated as the weighted averages of the logarithm for the estimated EC₅₀ for each TM cell strain in the study. The inverse of the variance for the estimates was used for the weight. The logarithms of the standard errors (SE) for the estimates were converted back to the original scale using the Taylor series expansion.

Effects of DEX and BOL-303242-X on Myocilin mRNA Expression

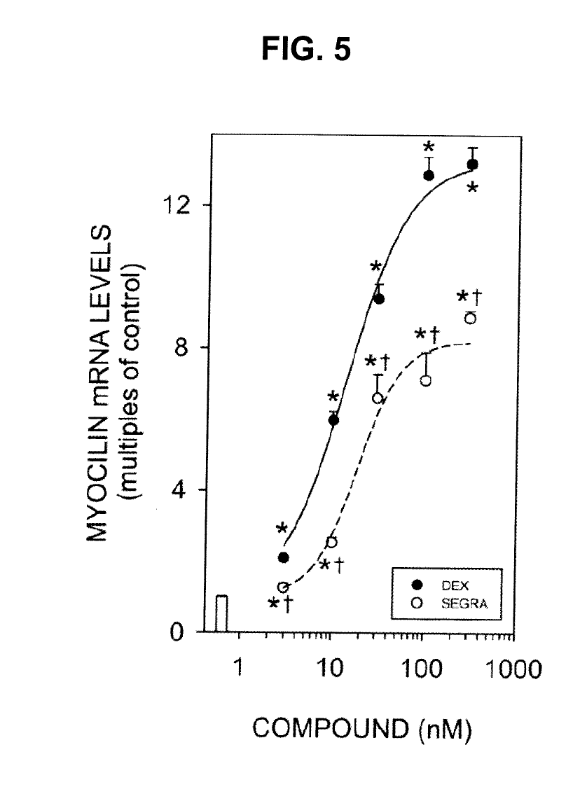

The effects of DEX and BOL-303242-X on myocilin mRNA expression in monkey TM are exemplified by the results shown in FIG. 5; data are from the same cell strain depicted in FIG. 4 (above). The patterns for expression of mRNA for myocilin were quite similar to those for protein, in terms of the dose-response to DEX vs. BOL-303242-X (FIG. 5 panel), also showing similar statistical significances to those observed for the protein levels. The BOL-303242-X qRT-PCR data again were indicative of the partial agonist nature of this agent, with significantly lower mRNA abundance values at all doses compared with DEX. Maximal efficacy, demonstrated at 300 nM for BOL-303242-X, was approximately 67% of that for DEX (FIG. 5). Regarding estimated EC₅₀s for all three drugs, there was excellent general correlation between the values both for myocilin protein and for mRNA abundance (Cf. Tables T-16 and T-17). Indeed, as previously shown with myocilin protein in Table T-15, the average (for n=4 strains) relative values for myocilin message were significantly lower for BOL-303242-X vs. DEX at both 100 and 300 nM (FIG. 5; solid and open symbols for DEX- and BOL-303242-X-treated cells, respectively).

TABLE T-17
Comparison of the potency of DEX and BOL-303242-X on
expression of myocilin mRNA in cultured monkey TM cells.
Compilation of data from two independent dose-response studies,
each using two monkey TM cell strains.

| | | | Coefficient of |
|---|---|---|---|
| | | 95% Confidence | Variation |
| Compound | EC₅₀ ± SE (nM)* | Limits for EC₅₀ | (%) |
| DEX | 14.66 ± 1.27 | 12.37-17.38 | 8.68 |
| BOL-303242-X | 20.75 ± 2.74 | 16.02-26.88 | 13.21 |

*The EC₅₀s presented were calculated as the weighted averages of the logarithm for the estimated EC₅₀ for each TM cell strain in the study. The inverse of the variance for the estimates was used for the weight. The logarithms of the standard errors (SE) for the estimates were converted back to the original scale using the Taylor series expansion.

### Effects of Drugs on Cultured Monkey TM Cells in the Resazurin Reduction Assay

There was no correlation of myocilin expression levels with general cell metabolic status, as a consequence of exposure to different concentrations of DEX or BOL-303242-X, nor did any drug treatments result in a loss of cell viability compared to vehicle controls, as determined by measuring chemical reduction of resazurin at the conclusion of the treatment periods (results not shown). The results suggest, then, that any increases or decreases observed in myocilin expression relative to control, induced by any of the drug treatment regimens, were not due to compromise of functional cell integrity.

Taken together, our results presented herein indicate that BOL-303242-X exhibits a full agonist profile as an anti-inflammatory agent and can have a more favorable therapeutic index than conventional GCs when used for the treatment of ocular diseases with an inflammatory component.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A method for treating or controlling an ocular inflammatory disease, condition, or disorder, comprising administering a composition comprising a DIGRA, a prodrug thereof, or a pharmaceutically acceptable salt or ester thereof to an affected eye of a subject in need of such treatment or control, wherein the DIA has Formula I 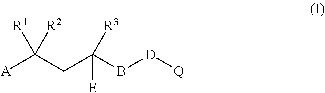
wherein A and Q are independently selected from the group consisting of unsubstituted and substituted aryl and heteroaryl groups, unsubstituted and substituted cycloalkyl and heterocycloalkyl groups, unsubstituted and substituted cycloalkenyl and heterocycloalkenyl groups, unsubstituted and substituted cycloalkynyl and heterocycloalkynyl groups, and unsubstituted and substituted heterocyclic groups; R¹ and R² are independently selected from the group consisting of hydrogen, unsubstituted C₁-C₁₅ linear or branched alkyl groups, substituted C₁-C₁₅ linear or branched alkyl groups, unsubstituted C₃-C₁₅ cycloalkyl groups, and substituted C₃-C₁₅ cycloalkyl groups; R³ is selected from the group consisting of hydrogen, unsubstituted C₁-C₁₅ linear or branched alkyl groups, substituted C₁-C₁₅ linear or branched alkyl groups, unsubstituted C₃-C₁₅ cycloalkyl and heterocycloalkyl groups, substituted C₃-C₁₅ cycloalkyl and heterocycloalkyl groups, aryl groups, heteroaryl groups, and heterocyclylic groups; B comprises a carbonyl, amino, divalent hydrocarbon, or heterohydrocarbon group; E is hydroxy or amino group; and D is absent or comprises a carbonyl group, —NH—, or —NR′—, wherein R′ comprises an unsubstituted or substituted C₁-C₁₅ linear or branched alkyl group; and wherein R¹ and R² together may form an unsubstituted or substituted C₃-C₁₅ cycloalkyl group; wherein DIGRA, a prodrug thereof, or a pharmaceutically acceptable salt or ester thereof is present in an amount effective to treat or control said ocular inflammatory disease, condition, or disorder; wherein the method provides a lower risk of inducing increased IOP than a method using a glucorticosteroid, and wherein said lower risk results from a lower production of myocilin from trabecular meshwork.

2. The method of claim 1, wherein said disease, condition, or disorder is selected from the group consisting of anterior uveitis, posterior uveitis, panuveitis, keratitis, conjunctivitis, vernal keratoconjunctivitis, atopic keratoconjunctiviti), corneal ulcer, corneal edema, sterile corneal infiltrates, anterior scleritis, episcleritis, blepharitis, and post-operative (or post-surgical) ocular inflammation resulting from procedures such as photorefractive keratectomy, cataract removal surgery, intraocular lens implantation, laser-assisted in situ keratomileusis (“LASIK”), conductive keratoplasty, radial keratotomy, dry eye, macular degeneration, macular edema, diabetic retinopathy, wet age-related macular degeneration, dry age-related macular degeneration, and glaucoma.

3. The method of claim 2, wherein the DIGRA has Formula I 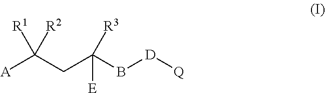
wherein A and Q are independently selected from the group consisting of aryl and heteroaryl groups substituted with at least a halogen atom, cyano group, hydroxy group, or C₁-C₁₀ alkoxy group; R¹, R², and R³ are independently selected from the group consisting of unsubstituted and substituted C₁-C₅ alkyl groups; B is a C₁-C₅ alkylene group; D is the —NH— or —NR′— group, wherein R′ is a C₁-C₅ alkyl group; and E is the hydroxy group.

4. The method of claim 2, wherein the DIGRA has Formula I 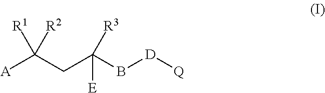
wherein A comprises a dihydrobenzofuranyl group substituted with a fluorine atom; Q comprises a quinolinyl or isoquinolinyl group substituted with a methyl group; R¹ and R² are independently selected from the group consisting of unsubstituted and substituted C₁-C₅ alkyl groups; B is a C₁-C₃ alkylene group; D is the —NH— group; E is the hydroxy group; and R³ comprises a trifluoromethyl group.

5. The method of claim 4, wherein the DIGRA has Formula II or III 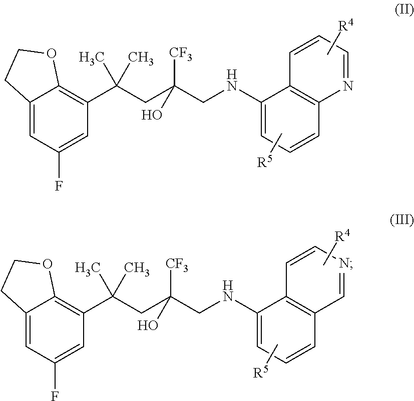
wherein R⁴ and R⁵ are independently selected from the group consisting of hydrogen, halogen, cyano, hydroxy, C₁-C₁₀ alkoxy groups, unsubstituted C₁-C₁₀ linear or branched alkyl groups, substituted C₁-C₁₀ linear or branched alkyl groups, unsubstituted C₃-C₁₀ cyclic alkyl groups, and substituted C₃-C₁₀ cyclic alkyl groups.

6. The method of claim 5, wherein the DIGRA has Formula IV

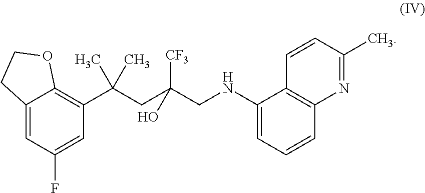

7. The method of claim 6, wherein said composition further comprises an anti-inflammatory agent is selected from the group consisting of NSAIDs, PPAR agonists, combinations thereof, and mixtures thereof.
